(19) United States
(12) Patent Application Publication
(10) Pub. No.: US 2015/0237796 A1
(43) Pub. Date: Aug. 27, 2015

(54) APPARATUS AND METHOD FOR LOCALIZED IRRIGATION AND APPLICATION OF FERTILIZERS, HERBICIDES, OR PESTICIDES TO ROW CROPS

(71) Applicants: ROBERT CELLI, STOCKTON, CA (US); DAVID CELLI, JR., STOCKTON, CA (US)

(72) Inventors: ROBERT CELLI, STOCKTON, CA (US); DAVID CELLI, JR., STOCKTON, CA (US)

(21) Appl. No.: 14/624,338

(22) Filed: Feb. 17, 2015

Related U.S. Application Data
(60) Provisional application No. 61/966,463, filed on Feb. 24, 2014.

(51) Int. Cl. A01C 23/00; G01N 21/27; A01G 25/16; G01N 33/24; A01G 25/00; A01C 23/04

(52) U.S. Cl. CPC A01C 23/007; A01G 25/00; A01C 23/047; A01G 25/16; G01N 33/24; G01N 21/27; G01N 2033/245

(57) ABSTRACT

An apparatus and method are provided for selectively providing delicate nascent plants with a predetermined volume of water containing fertilizer, pesticide, or herbicide to row crops in agriculture operations. The hydraulic apparatus, together with certain electronic controls, delivers aliquots of aqueous solution rapidly, yet under low pressure, thereby ensuring that delicate nascent plants are not damaged by high-pressure flows and also ensuring that bare soil is not subject to erosion. The on/off control of the hydraulic apparatus is provided by means of light emitters.

### CROSS-REFERENCE TO RELATED APPLICATIONS

This application claims the benefit of U.S. Provisional Application No. 61/966,463, filed Feb. 24, 2014.

### FIELD OF THE INVENTION

The present invention relates in general to agricultural equipment. In particular, the present invention relates to an apparatus and a method for selectively providing delicate nascent plants with a predetermined volume of water containing fertilizer, pesticide, or herbicide to row crops in agriculture operations.

### BACKGROUND OF THE INVENTION

The following description is not an admission that any of the information provided herein is prior art or relevant to the present invention, or that any publication specifically or implicitly referenced is prior art. Any publications cited in this description are incorporated by reference herein. Where a definition or use of a term in an incorporated reference is inconsistent or contrary to the definition of that term provided herein, the definition of that term provided herein applies and the definition of that term in the reference does not apply.

The traditional method for delivering a known volume of water containing fertilizers, herbicides or pesticides (hereinafter called “aqueous solution”) to row crops is to use a means of direct application of irrigation solution towed behind a tractor. As the tractor traverses a row of crops, the irrigation apparatus can be turned on and off so as to cause a predetermined volume of aqueous solution to be delivered to a spatially localized region surrounding individual plants. Attempts to speed up the process by utilizing faster tractor speeds and employing electronically controlled irrigation valves have not produced satisfactory results because faster tractor speeds require higher flow rates which cause damage when applied directly to individual plants during a brief period of time. The limiting factor in the use of automated irrigation technology has been the unavoidable damage to the plants and topsoil due to the intermittent high velocity flows emanating from high-pressure irrigation apparati. Put simply, high velocity flow damages fragile vegetative structures and causes local soil erosion.

The historical apparatus for delivering a predetermined volume of solution upon crops has been the use of a hand-held bucket, watering can, or similar vessel. A predetermined volume of aqueous solution is discharged upon a plant with low flow over a prolonged period of time or, in the alternative, a high flow over a lesser period of time, depending upon the angle with which the bucket or watering can is held during its use. The use of a bucket or watering can is impractical for watering row crops at high production rates because the cycle time between adjacent plants encountered as the tractor traverses row crops at high speeds is shorter than the cycle time for periodic filling plus emptying of a bucket or watering can.

The ideal method of delivery of aqueous solution to crops should accommodate the need for greater reactor speed, predetermined aqueous solution volumes, low flow velocities of effluent delivered to individual plants, localization of deposition, and rapid cycle of times. Thus, there exits a need for a system that rapidly delivers a predetermined amount of aqueous solution with a low velocity on a spatially localized area surrounding individual row crops in a brief period of time.

The customary apparatus and method for applying water, fertilizers, herbicides and pesticides to delicate nascent row crops is to employ field workers to walk the rows of crops and apply water and fertilizers and herbicides and pesticides by means of hand-controlled irrigation apparati. The result is: (i) an uneven distribution of water and fertilizers and herbicides and pesticides; (ii) application onto bare ground where it is useless and contributes to soil erosion and scour; (iii) some plants are missed altogether whilst others are sprayed twice; (iv) ineffective use of fertilizers, herbicides and pesticides; (v) overspray that can adversely affect the health of farm workers; and (vi) uneconomical use of increasingly expensive farm labor.

Irrigation by means of hand-controlled, tractor-mounted irrigation rigs is the conventional method of applying fertilizers and pesticides and herbicides. However, when irrigation is applied in such a manner, most of the effluent is wasted because it is deposited on vacant ground where the nutritional or medicinal additives have no effect. Moreover, fertilizers, pesticides and herbicides may have highly undesirable effects on the environment, for example, by causing contamination of ground water or causing chemical burning of different crops in adjacent fields during floods. Thus, a need exists to reduce both fertilizer and pesticide and herbicide cost and environmental impact by selectively irrigating plants (only).

In many situations, it is necessary to distinguish one type of object from another. While this is essential in such diverse areas as manufacturing, data processing and mail delivery, object differentiation is particularly important in agriculture. For example, the ability to distinguish plants from bare soil is essential to enable plants to be given water, nutrients, herbicides and pesticides that would otherwise be wasted on bare soil.

Methods for optically distinguishing between soil and plants are currently known in the art. However, many of these methods use devices that rely on natural sunlight to create a reflected image. Thus, the devices cannot operate at night, and are seriously impaired under cloudy conditions, or even when operated in shadows. Other methods for optically distinguishing between soil and plants use devices that rely on an artificial white light source to create the reflected image. However, under normal (i.e. sunny) operating conditions, this artificial light source must compete with the sun that is thousands of times brighter and constantly changing in brightness and spectral distribution. Therefore, either method (i.e., using natural sunlight or an artificial white light source) fails to reliably provide an accurate wavelength signature of objects in the field of view of the device sufficient to optically recognize all individual plants within the visual field of the optical sensor. Indeed, applications for these methods have been limited to low-till or no-till field crops where this lack of precision can be tolerated. However, row crop applications require a high degree of accuracy in optically identifying and localizing individual plants. Thus, a need exists for a device that provides accurate optical sensing of nascent plants and efficient irrigation, fertilization and treatment of individual plants.

The use of optical control apparati has not been practical in controlling irrigation apparati because the output from optical control apparati is low voltage and low amperage electrical power.

Methods for dispensing water, fertilizers, pesticides and herbicides are still largely based on hand-controlled, tractor-mounted irrigation rigs. The primary difficulty with such irrigation methods is that they necessarily rely upon intermittent high velocity flows. An intermittent high-velocity flow results in unwanted soil erosion and is likely to result in structural damage to delicate nascent plants. For this reason, any irrigation apparatus that uses intermittent high-velocity flows is contraindicated despite the obvious simplicity of such a system.

### SUMMARY OF THE INVENTION

Various objects, features, aspects and advantages of the inventive subject matter will become more apparent from the following detailed description of exemplary embodiments, along with the accompanying figures in which like numerals represent like components.

In one embodiment, the present invention provides: an electronically-controlled apparatus for applying an aqueous solution to row crops comprising an aqueous solution tank; a master valve comprising a 3-way valve operably interconnected to the aqueous solution tank; a pump operably interconnected to the aqueous solution tank; a compressor operably interconnected to the pump; a pressure reservoir operably interconnected to the compressor; at least one irrigation valve operably interconnected to the compressor; at least one precision hydraulic applicator; at least one low-pressure jet integrated with the precision hydraulic applicator; an input hose having a progressively increasing diameter, wherein the input hose is located between the output of the at least one irrigation valve and the input of the precision hydraulic applicator; at least one timer; at least one relay; an electronic control subsystem comprising a first emitter and a second emitter, at least one photo detector a phase detector, and a controller, wherein the optical detector, the phase detector, and the controller are all electrically interconnected, wherein the at least one irrigation valve is electrically interconnected to the at least one timer, the at least one relay, and the electronic control subsystem, and a power source electrically interconnected to the electronic control subsystem, the pump, the timer, the relay, the first emitter, the second emitter, and the compressor. In yet another embodiment, the precision hydraulic applicator comprises at least 2 standpipes.

In a further embodiment, the standpipes are arranged in series, in parallel, or any combination thereof.

In another embodiment, at least one low-pressure jet has a muzzle velocity of about 7 feet per second.

In still another embodiment, the aqueous solution comprises water, fertilizer, pesticide, herbicide, or any combination thereof.

In another embodiment, the invention provides an apparatus wherein the first emitter emits radiation at a wavelength of about 670 nm, and the second emitter emits radiation at a wavelength of about 750 nm.

In a further embodiment, at least one low-pressure jet comprises a manifold having a plurality of low-pressure jets.

In yet another embodiment, the precision hydraulic applicator comprises a pipe having a diameter from about 2 to 6 inches.

In yet another embodiment, the present invention provides an electronically-controlled apparatus for applying an aqueous solution to row crops comprising an aqueous solution tank; a master valve comprising a 3-way valve operably interconnected to the aqueous solution tank; a pump operably interconnected to the aqueous solution tank; a compressor operably interconnected to the pump; a pressure reservoir operably interconnected to the compressor; a plurality of irrigation valves operably interconnected to the compressor; a plurality of precision hydraulic applicators, wherein each of the precision hydraulic applicators further comprises a manifold having a plurality of low-pressure jets integrated with each of the respective precision hydraulic applicators; a plurality of input hoses having a progressively increasing diameter, wherein each of the input hoses is located between the output of a corresponding irrigation valve and the corresponding input of a precision hydraulic applicator; a plurality of timers; a plurality of relays; an electronic control subsystem comprising a first emitter and a second emitter; a first photo detector; a second photo detector; a phase detector; and a controller; wherein each of the photo detectors, the phase detector, and the controller are all electrically interconnected; wherein each of the irrigation valves is electrically interconnected to a corresponding timer, a corresponding relay, and the electronic control subsystem; and a power source electrically interconnected to: the electronic control subsystem, the pump, the timers, the relays, the first emitter, the second emitter, and the compressor.

In still another embodiment, the present invention provides an electronically-controlled apparatus for applying an aqueous solution to row crops comprising an aqueous solution tank; a master valve comprising a 3-way valve operably interconnected to the aqueous solution tank; a pump operably interconnected to the aqueous solution tank; a compressor operably interconnected to the pump; a pressure reservoir operably interconnected to the compressor; at least one irrigation valve operably interconnected to the compressor; at least one precision hydraulic applicator; a manifold comprising a plurality of low-pressure jets integrated with the precision hydraulic applicator; an input hose having a progressively increasing diameter, wherein the input hose is located between the output of the at least one irrigation valve and the input of the precision hydraulic applicator; at least one timer; at least one relay; an electronic control subsystem comprising a first emitter and a second emitter; at least one photo detector; a phase detector; and a controller; wherein the optical detector, the phase detector, and the controller are all electrically interconnected; wherein the at least one irrigation valve is electrically interconnected to the at least one timer, the at least one relay, and the electronic control subsystem; and a power source, wherein the power source is electrically interconnected to: the electronic control subsystem, the pump, the at least one timer, the at least one relay, the first emitter, the second emitter, and the compressor.

In a further embodiment, the present invention provides a method of electronically and rapidly delivering an aqueous solution to a localized annulus of ground surrounding a plant, the method comprising the steps of (a) pumping the aqueous solution from a tank through a first pipe; (b) reducing the hydrostatic pressure and velocity of the aqueous solution by continuing to flow the aqueous solution into a second pipe having a greater diameter than the first pipe; (c) maintaining laminar flow of the aqueous solution simultaneously with step (b); (d) streaming a plurality of aliquots of the aqueous solution in an upward direction from the output of the second pipe into the center of) a standpipe; (e) terminating the streaming of step (d); and (f) delivering the aqueous solution at a low velocity from the standpipe to the target area with a specified wetting pattern.

In yet another embodiment, the invention provides streaming a plurality of aliquots of the aqueous solution in an upward direction from the output of the second pipe into the center of a standpipe as herein described and which further comprises suspending progressive columns of aqueous solution in the standpipe, or streaming alternatively comprises injecting a plurality of simultaneous streams of the aqueous solution from a series of low pressure jets into the standpipe.

In another embodiment, the present invention provides a method as herein described, wherein steps (a) to (f) as provided are completed within a total cycle timeframe of about 1 second. In still another embodiment, the present invention provides a method as herein described, wherein step (f) as herein described produces a resulting oval wetting pattern of about 3 inches by 6 inches.

In a further embodiment, the present invention provides a method as herein described, wherein the low velocity in step (f) is less than about 14 feet per second.

In another embodiment, the low velocity in step (f) is an average of about 13 feet per second.

In yet another embodiment, the present invention provides an aqueous solution that comprises water, fertilizer, pesticide, herbicide, or any combination thereof.

In a further embodiment, the present invention provides a method of automatically sensing and distinguishing a plant from soil, the method comprising the steps of (a) emitting radiation of a first wavelength from a first emitter; (b) emitting radiation of a second wavelength from a second emitter; (c) modulating the first and second emitters at a high rate of speed; (d) shifting the modulation of the first emitter by approximately 90 degrees relative to the second emitter; (e) focusing the first and second emitters on a target to reflect the first and second emitter wavelengths; (f) using a photo receptor to intercept the reflected radiation wavelengths from step (e); (g) calculating a ratio value from step (f) of the first and second reflected wavelengths; (h) converting the ratio value of step (g) to a phase; (i) comparing the phase of step (h) to an initial reference phase of the first or said second emitter; and (j) processing the output of step (i) via a digital controller to electronically determine the presence of a plant or soil.

In another embodiment, the invention provides a method wherein the first wavelength is about 670 nm and the second wavelength is about 750 nm.

### BRIEF DESCRIPTION OF THE DRAWINGS

FIG. 1 shows schematically one embodiment of the hydraulic apparatus and electronic controls in accordance with the invention.

FIG. 2 illustrates one embodiment of an on/off photo-detector type of electronic controller used to regulate the on/off flow of aqueous solution into the hydraulic apparatus in accordance with the present invention.

FIG. 3 illustrates a graph of representative curves of the reflectance of a plant and of soil for various wavelengths.

FIG. 4 illustrates one embodiment of a hose barb adapter (Part A) where one end is a 1¼ inch barb fitting and the other end is a 1½ inch male national pipe thread taper (hereinafter called “NPT”) flared and threaded fitting in accordance with the invention.

FIG. 5 illustrates one embodiment of a pipe adapter (Part B) where one end is a 1½ inch female NPT threaded fitting and the other end is a 1½ inch female nominal pipe size (hereinafter “NPS”) slip socket fitting in accordance with the invention.

FIG. 6 illustrates an exemplary embodiment of a 1½ inch NPS pipe (Part C) in accordance with the invention.

FIG. 7 illustrates an exemplary embodiment of a pipe adapter (Part D) where one end is a 1-½ inch female slip socket fitting and the other end is a 2-inch NPS pipe in accordance with the invention.

FIG. 8 illustrates an exemplary embodiment of a pipe adapter (Part E) where one end is a 2-inch female slip socket fitting and the other end is a 3-inch NPS pipe in accordance with the invention.

FIG. 9 illustrates an exemplary embodiment of a pipe elbow (Part F) of 90 degrees where both ends are a 1½ inch female slip socket fitting in accordance with the invention.

FIG. 10 illustrates an exemplary embodiment of a pipe tee (Part G) with 3-inch female slip socket fittings on all openings in accordance with the invention.

FIG. 11 illustrates an exemplary embodiment of a 3-inch NPS pipe (Part H) in accordance with the invention.

FIG. 12 illustrates an exemplary embodiment of a pipe coupling (Part I) where both ends are 3-inch female slip socket fittings in accordance with the invention.

FIG. 13 illustrates an exemplary embodiment of a pipe coupling (Part J) where both ends are 3-inch female slip socket fittings in accordance with the invention.

FIG. 14 illustrates an exemplary embodiment of a 3-inch NPS pipe (Part K) in accordance with the invention.

FIG. 15 illustrates an exemplary embodiment of a 3-inch NPS pipe (Part L) in accordance with the invention.

FIG. 16 illustrates an exemplary embodiment of a 3-inch NPS pipe (Part M) in accordance with the invention.

FIG. 17 illustrates an exemplary embodiment of the assembly of Parts A (FIG. 4), B (FIG. 5) and C (FIG. 6) in accordance with the invention. The male threaded end of Part A (FIG. 4) is connected to the female threaded end of Part B (FIG. 5). The female slip socket end of Part B (FIG. 5) is connected to Part C (FIG. 6).

FIG. 18 illustrates an exemplary embodiment of the alignment for which Parts D (FIG. 7), E (FIG. 8), and G (FIG. 10) are connected in accordance with the invention.

FIG. 19 illustrates an exemplary embodiment of the assembly of Parts D (FIG. 7), E (FIG. 8) and G (FIG. 10) in accordance with the invention. The pipe end of Part D (FIG. 7) is connected to the female slip socket end of Part E (FIG. 8). The pipe end of Part E (FIG. 8) is connected to the female slip socket connection of Part G (FIG. 10) perpendicular to the transverse ends.

FIG. 20 illustrates an exemplary embodiment of the assembly of Parts A (FIG. 4), B (FIG. 5), C (FIG. 6), D (FIG. 7), E (FIG. 8) and F (FIG. 9) in accordance with the invention. The pipe end of Part D (FIG. 7) is connected to the female slip socket end of Part E (FIG. 8). The pipe end (non-barbed) of Assembly ABC (FIG. 17) is connected to the female slip socket end of Part D (FIG. 7). Part C (FIG. 6) is long enough to be fastened to Part B (FIG. 5) and extend beyond the pipe end of Part E (FIG. 8) and into Part F (FIG. 9).

FIG. 21 illustrates an exemplary embodiment of the assembly of Parts A (FIG. 4), B (FIG. 5), C (FIG. 6), D (FIG. 7), E (FIG. 8), F (FIG. 9) and G (FIG. 10). Assembly ABCDEF (FIG. 20) is connected to Part G (FIG. 10) via the connection of the pipe end of Part E (FIG. 20) into the female slip socket connection of Part G (FIG. 21).

FIG. 22 illustrates an exemplary embodiment of the assembly of Parts H (FIG. 11), G (FIG. 10) and K (FIG. 14) in accordance with the invention. Part H (FIG. 11) is connected to either one of the transverse ends of Part G (FIG. 10). Part K (FIG. 14) is connected to the other transverse end of Part G (FIG. 10).

FIG. 23 illustrates an exemplary embodiment of the assemblage of Parts K (FIG. 14), J (FIG. 13) and M (FIG. 16) in accordance with the invention. One end of Part L (FIG. 12) is connected to one end of Part I (FIG. 9). One end of Part H (FIG. 8) is connected to the other end of Part I (FIG. 9).

FIG. 24 illustrates an exemplary embodiment of the assembly of Parts H (FIG. 11), I (FIG. 12) and L (FIG. 15) in accordance with the invention. One end of Part K (FIG. 11) is connected to one end of Part J (FIG. 10). One end of Part M (FIG. 13) is connected to the other end of Part J (FIG. 10).

FIG. 25 illustrates an exemplary embodiment of the assembly of Parts A (FIG. 1), B (FIG. 2), C (FIG. 3), D (FIG. 4), E (FIG. 5), F (FIG. 6), G (FIG. 7), H (FIG. 8), I (FIG. 9), J (FIG. 10), K (FIG. 11), L (FIG. 12), M (FIG. 13) and N (FIG. 14) in accordance with the invention. Assembly HIL (FIG. 23) is connected to Assembly ABCDEFG (FIG. 21) by means of the connection of the open pipe end of Part H (FIG. 23) into Part G (FIG. 21) where the open end of Part F (FIG. 21) is located. Assembly KJM (FIG. 24) is connected to Assembly ABCDEFG (FIG. 21) by means of the connection of the open end of Part K (FIG. 24) into remaining portal of Part G (FIG. 21).

FIG. 26 illustrates an exemplary embodiment of the assembly of Parts A (FIG. 1), B (FIG. 2), C (FIG. 3), D (FIG. 4), E (FIG. 5), F (FIG. 6), G (FIG. 7), H (FIG. 8), I (FIG. 9), J (FIG. 10), K (FIG. 11), L (FIG. 12), M (FIG. 13) and N (FIG. 14) into assembly ABCDEFGHIJKLMN into what shall hereinafter be termed the “standpipe” in accordance with the invention.

FIG. 27 illustrates an exemplary embodiment of the overall schematic where two standpipes are used in series in accordance with the invention. In this embodiment, the timers are wired in parallel.

FIG. 28 illustrates an exemplary embodiment of the use of two standpipes used in parallel in accordance with the invention. In this embodiment, the entire assembly is towed behind a tractor.

FIG. 29 illustrates an exemplary embodiment of the timeline of the sequence of events starting with the detection of a plant by optical means through one complete duty cycle using the apparatus that is the subject of this invention.

FIG. 30 illustrates an exemplary embodiment of the timeline of the sequence of events (as in FIG. 29) where two standpipes are used in series in accordance with the invention.

FIG. 31 illustrates an exemplary embodiment of the timeline of one complete duty cycle according to the invention.

FIG. 32 illustrates an exemplary embodiment of the overall geometry of the standpipe in use according to the invention.

FIG. 33 illustrates an exemplary embodiment of the spatial pattern of wetting upon the ground as a result of one complete duty cycle according to the invention.

FIGS. 34 through 56 depict another exemplary embodiment of the hydraulic apparatus of the invention, wherein the input of aqueous solution is direct upwards into the standpipe by means of individual 3/16-inch diameter jets of solution delivered by means of a manifold.

FIG. 34 illustrates an exemplary embodiment of a hose barb adapter (Part N) where one end is a 1-inch barb fitting and the other end is a 1 inch male national pipe thread taper (hereinafter called “NPT”) flared and threaded fitting according to the invention.

FIG. 35 illustrates an exemplary embodiment of a 4-inch×4-inch×-inch Reducing Tee (S×S×FPT) PVC Schedule 40 Pipe Fitting (Part O) according to the invention.

FIG. 36 illustrates an exemplary embodiment of a length of 1½ inch nominal diameter Schedule 40 PVC Pipe with a plurality of 3/16-inch diameter holes drilled diagonally upward through the wall of the Schedule 40 PVC Pipe at an angle of approximately forty degrees)(40° plus or minus five degrees)(5° (Part P) in accordance with the invention. In this embodiment, the number of 3/16-inch diameter holes can vary between four (4) holes and twelve (12) holes. The 3/16-inch diameter holes are drilled approximately at mid-way along the length of 1½ inch nominal diameter Schedule 40 PVC Pipe; however, the exact location is not critical. The 1½ inch nominal diameter Schedule 40 PVC Pipe can vary in length from 12 inches to 14 inches.

FIG. 37 illustrates an exemplary embodiment of a segment of 1½ inch nominal diameter Schedule 80 PVC (S×S) Coupling with a plurality of 3/16-inch diameter holes drilled diagonally upward through the wall of the Schedule 40 PVC Pipe at an angle of approximately forty degrees) (40° plus or minus five degrees)(5° (Part Q) according to the invention. The number of 3/16-inch diameter holes can vary between four (4) holes and twelve (12) holes. The holes in Part Q must be aligned with the holes in Part P. The 1½ inch nominal diameter Schedule 80 PVC (S×S) Coupling can vary in length from ¾-inch to 1 inch. The inside diameter of Part Q must be free of burrs or lips or other obstructions that might otherwise prevent Part P from fitting into and sliding through Part Q. In other words, the inside diameter of Part Q must be the exact same size as the outside diameter of Part P. The number of 3/16-inch diameter holes can vary between four (4) holes and twelve (12) holes; however the number of holes in Part Q must be the same as the number of holes in Part P. The relationship between Part P and Part Q is shown in FIG. 45. It is advisable to glue Part P and Part Q together using PVC cement to form the Assembly PQ before drilling the 3/16 inch holes through Part P and Part Q (i.e., Assembly PQ). Fastening Part P and Part Q before drilling ensures proper alignment of the 3/16-inch diameter holes.

FIG. 38 illustrates an exemplary embodiment of a 3-inch to 1½ inch Schedule 40 PVC Reducer Bushing (Part R) according to the present invention. This is one of three (3) such 3 inch to 1½ inch reducer bushings used to make the hydraulic apparatus in this invention. This particular 3-inch to 1½ inch reducer bushing (Part R) is oriented with the larger diameter in the superior position and the smaller diameter in the inferior position.

FIG. 39 illustrates an exemplary embodiment of a 3-inch to 1½ inch Schedule 40 PVC Reducer Bushing (Part S) in accordance with the invention. This is one of three (3) such 3-inch to 1½ inch reducer bushings used to make the hydraulic apparatus in this invention. This particular 3-inch to 1½ inch reducer bushing (Part S) is oriented with the smaller diameter in the superior position and the larger diameter in the inferior position.

FIG. 40 illustrates an exemplary embodiment of a 3-inch to 1½ inch Schedule 40 PVC Reducer Bushing. (Part T) in accordance with the invention. This is one of three (3) such 3-inch to 1½ inch reducer bushings used to make the hydraulic apparatus in this invention. This particular 3-inch to 1½ inch reducer bushing (Part T) is oriented with the larger diameter in the superior position and the smaller diameter in the inferior position.

FIG. 41 illustrates an exemplary embodiment of a 4-inch to 3-inch Schedule 40 PVC Bell Reducer (Part U) according to the invention. The 4-inch to 3-inch Schedule 80 PVC Bell Reducer (Part U) is oriented with the larger diameter in the superior position and the smaller diameter in the inferior position.

FIG. 42 illustrates an exemplary embodiment of a segment of 4 inch nominal diameter Schedule 40 PVC Pipe (Part V) according to the invention. The length of the 4-inch nominal diameter Schedule 40 PVC Pipe is such that the volume inside the pipe segment is approximately ½ gallon. In other words, the 4-inch nominal diameter Schedule 40 PVC Pipe has a length of 12½ inches (minimum) to 13 inches (maximum). The 4-inch nominal diameter Schedule 40 PVC Pipe (Part V) functions as a temporary reservoir for one aliquot of solution to be deposited during each working cycle. The 4-inch nominal diameter Schedule 40 PVC Pipe (Part V) is oriented vertically.

FIG. 43 illustrates an exemplary embodiment of a 4-inch nominal diameter Schedule 40 PVC Cap (Part W) according to the invention. A plurality of ⅛-inch diameter air ventilation holes is drilled into the cap (Part W). The ventilation holes permit air to escape from 4-inch nominal diameter Schedule 40 PVC Pipe (Part V) during the filling of the reservoir during the working cycle. The number of air ventilation holes can range from four (4) holes to eight (8) holes.

FIG. 44 illustrates an exemplary embodiment of the assembly of Part N and Part O and Part R to form Assembly NOR in accordance with the invention. Part N is screwed into Part O. Part R is permanently fastened to Part O by means of PVC cement.

FIG. 45 illustrates an exemplary embodiment of the relationship between Part P and Part Q according to the invention. It is advisable to glue Part P and Part Q together using PVC cement to form the Assembly PQ before drilling the 3/16 inch holes through Part P and Part Q (i.e., Assembly PQ). Fastening Part P and Part Q before drilling the 3/16-inch holes ensures proper alignment of the 3/16-inch diameter holes.

FIG. 46 illustrates an exemplary embodiment of how Assembly NOR and Assembly PC fit together to form Assembly NOPRQ according to the invention. The space between the outside of Assembly PC and the interior of Assembly NOR serves as a manifold that allows solution to flow through the 3/16-inch diameter holes drilled in Assembly PC and be directed upward so as to create jets of solution upwards within the 1½ inch pipe (Part P).

FIG. 47 illustrates an exemplary embodiment of the relationship between the reducing bushing (Part S) and the 1½ inch pipe (Part P) that forms an integral part of Assembly NOPRQ according to the invention.

FIG. 48 illustrates an exemplary embodiment of how the reducing bushing (Part S) slides over the 1½ inch pipe (Part P) to form the Assembly NOPRQS according to the invention. The reducing bushing (Part S) is fastened to the 1½ inch pipe (Part P) by means of PVC cement.

FIG. 49 illustrates an exemplary embodiment of how the larger end of the Schedule 40 PVC 3-inch to 1½ inch Reducer Bushing (Part T) is permanently fastened to the inside surface of the Schedule 40 PVC 4-inch to 3-inch Reducer Bushing (Part U) to form Assembly TU according to the invention. The 3-inch to 1½ inch Reducer Bushing (Part T) is fastened permanently to the inside of the Schedule 40 PVC 4-inch to 3-inch Reducer Bushing (Part U) by means of PVC cement.

FIG. 50 illustrates an exemplary embodiment of how Assembly TU is fastened permanently to Assembly NOPRQS to form Assembly NOPRQSTU by means of PVC cement according to the invention.

FIG. 51 illustrates an exemplary embodiment of the relationship between Part V and Part U according to the invention.

FIG. 52 illustrates an exemplary embodiment of how the reducer bushing (Part V) and the 4-inch diameter standpipe (Part U) are fastened permanently by means of PVC cement according to the invention.

FIG. 53 illustrates an exemplary embodiment of the relationship between the 4-inch cap (Part W) and the 4-inch diameter standpipe (Part V) according to the invention.

FIG. 54 illustrates an exemplary embodiment of how the 4-inch cap (Part W) is attached to the 4-inch diameter standpipe (Part V) according to the invention.

FIG. 55 illustrates an exemplary embodiment of the relationship between Assembly NOPRQS and Assembly TUVW and how they fit together to form Assembly NOPRQS TUVW according to the invention. Assembly TUVW slides over the 1½ inch diameter pipe (Part P) so as to close the gap between the 3 inch to 1½ inch bell reducer (Part T) and the other 3 inch to 1½ inch bell reducer (Part S).

FIG. 56 illustrates an exemplary embodiment of the final Assembly NOPRQSTUVW according to the invention.

FIG. 57 illustrates an exemplary embodiment of the final Assembly NOPRQSTUVW in its ready-to-use configuration according to the invention.

### DETAILED DESCRIPTION OF THE INVENTION

The present method of delivery of solution to row crops accommodates the need for greater tractor speed, predetermined aqueous solution volumes, low flow velocities of effluent delivered to individual plants, localization of the deposition, and rapid cycle times. The invention is directed to an apparatus and a method for selectively providing delicate nascent plants with a predetermined volume of aqueous solution. In one embodiment, the hydraulic apparatus, together with certain electronic controls, delivers aliquots of aqueous solution rapidly, yet under low pressure, thereby ensuring that delicate nascent plants are not damaged by high-pressure flows and also ensuring that bare soil is not subject to erosion. The on/off control of the hydraulic apparatus is provided by means of light emitters described herein. The streaming of the aliquots along the approximate center is still effective in the present invention, while a variation of approximately 10% from true center achieves the same result.

To provide selective irrigation on plants and not on bare soil, two light (radiation) emitters, powered by an internal power source, are modulated to switch on and off at very high speeds. Each emitter emits radiation of a different emitter wavelength. The on/off modulation of one emitter is phase shifted by approximately 90° with respect to the modulation of the other emitter. The pair of emitters is focused on a particular spot on the ground. The light beams, which are provided by the emitters, are reflected off a plant or the soil and are intercepted by a photo-detector. Because plants have a characteristic spectral reflectance in regions of the electromagnetic spectrum which can be discriminated from the spectral reflectance of the background earth, the relative amplitudes of the reflected radiation at the two-emitter wavelengths varies depending on whether the radiation is reflected off a plant or the soil. A ratio of the radiation at the two-emitter wavelengths received by the photo-detector is converted to a phase. This phase is compared to an initial reference phase of the modulation of one of the emitters. A controller uses this phase information to determine the presence or absence of a plant and then irrigates the plant. Further electronic control elements are utilized to operate hydraulic valves.

In one embodiment, this invention provides for the use of timers and load relays to boost the voltage and amperage of the electrical power so as to be available to operate heavy-duty irrigation valves. One of ordinary skill in the art will be able to readily understand how timers and load relays are used to boost the voltage and electrical power. For example, see U.S. Pat. No. 2,831,981A.

The use of the venerable standpipe has been coupled in this invention with a modern electronic control system that results in an apparatus and method for localized delivery of aqueous solution under conditions of low-flow velocity flows and in a localized area of application surrounding individual delicate nascent plants. Furthermore, this method works at production rates that exceed what can be achieved by hand-controlled irrigation methods.

As used herein, and unless the context dictates otherwise, the terms “irrigation valve”, “control valve” and “solenoid valve”, may be used interchangeably.

As used in the description herein and throughout the claims that follow, the meaning of “a,” “an,” and “the” includes plural reference unless the context clearly dictates otherwise. Also, as used in the description herein, the meaning of “in” includes “into” and “on” unless the context clearly dictates otherwise.

As used herein, the term “about” in conjunction with a numeral refers to a range of that numeral starting from 10% below the absolute of the numeral to 10% above the absolute of the numeral, inclusive.

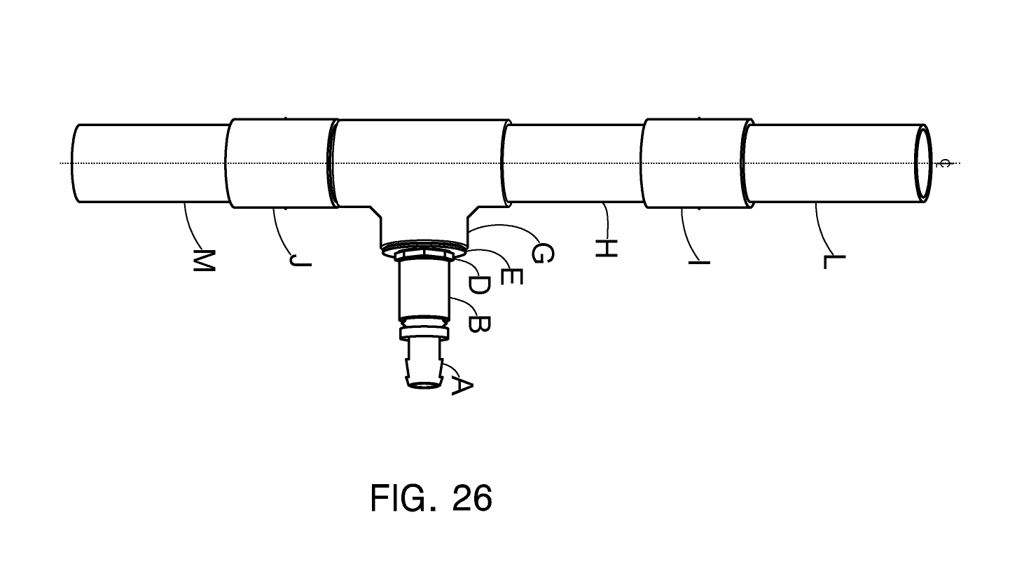

In an exemplary embodiment, the apparatus comprises a length of vertical standpipe of 3-inch nominal diameter, uncapped at both ends, into which an intermittent low pressure jet of solution is directed upwards by means of a 1½ inch nominal diameter discharge pipe located coaxially to the standpipe and located near the bottom end of the standpipe (see FIG. 26). In an embodiment, two or more standpipes may be used in parallel, in series, or combinations thereof. In one embodiment, the standpipe is open to the air on the bottom as well as on the top.

Initially, the standpipe is empty. The working cycle commences when a solution is discharged under moderate pressure oriented upward from a location near the bottom end of the standpipe. As more and more solution is discharged upwards under moderate pressure into the center of the standpipe and aligned coaxially with the standpipe, the initial solution deposited in the standpipe is temporarily prevented from flowing downward and outward through the open bottom of the standpipe by the momentum transfer delivered to the growing column of solution within the standpipe by the later jet of solution directed vertically upward. The streaming of the aliquots along the approximate center is still effective in the present invention, while a variation of approximately 10% from true center achieves the same result. The instant the upward jet of solution is terminated, the accumulated column of solution in the standpipe begins to fall downward and outward through the open bottom of the standpipe due to the effect of gravity. A predetermined volume of solution may thus be deposited periodically on a localized annulus of ground surrounding a plant whilst the tractor moves along the row of crops towing this irrigation apparatus behind.

The working cycle can be described mathematically as follows.

At time t=0, low velocity laminar flow of water plus herbicide and/or pesticide is turned on by an irrigation valve controlled electronically. The on/off value is controlled by means described below.

The on/off irrigation value requires 20 milliseconds (i.e., 0.020 seconds) for complete actuation.

At time t=0.020 seconds, aqueous solution flows from a pressure reservoir held at a constant pressure of 60 psi through a 1-inch diameter flexible pressure hose. The 1-inch diameter flexible pressure hose is attached to a 1¼ inch PVC pipefitting. Immediately downstream, the 1¼ inch PVC pipefitting is connected to a 1½ inch diameter right angle)(90° elbow made of PVC plastic pipe. Stepping up the pipe diameter in a sequence of two discrete steps, namely (i) from 1-inch diameter to 1¼ inch diameter; and (ii) from 1¼ inch diameter to 1½ inch diameter, accomplishes the objective of reducing the hydrostatic pressure and reducing the velocity of the solution to 7 feet per second while maintaining laminar fluid flow conditions. The resultant jet of aqueous solution is directed upwards from the orifice of the 1½ inch diameter right angle)(90° elbow PVC pipefitting and is directed coaxially into the center of the open-ended 3-inch diameter standpipe with a muzzle velocity of 7 feet per second.

Consider aliquots of aqueous solution of known but arbitrary volume, say Vᵢ=0.001 cubic feet=1.728 cubic inches=0.9575 fluid ounces, discharged from the 1½ inch diameter PVC pipe directed upwards and coaxially into the open-ended 3-inch diameter standpipe.

At the moment the aqueous solution leaves the 1½ inch diameter discharge pipe and enters the 3-inch diameter standpipe (i.e., at time t=0.020 seconds), its upward velocity is approximately 7 feet per second. The jet of aqueous solution is directed upwards, coaxial to the standpipe. The jet of aqueous solution is located within the hollow interior of the 3-inch standpipe (see FIG. 26).

Given its muzzle velocity of 7 feet per second, the aqueous solution jet reaches a maximum height given by the following equation.

$${X}_{max} = \frac{{( {v}_{o} )}^{2}}{2 g}$$

where

Xₘₐₓ=the maximum height of the jet of aqueous solution above the orifice of the 1½ inch diameter discharge pipe;

g=32 ft per sec² (acceleration due to gravity);
v₀=7 ft per sec (initial upward velocity of the aqueous solution jet).

Therefore, the maximum height of the aqueous solution jet is given by the equation.

$${X}_{max} = \frac{{( {v}_{o} )}^{2}}{2 g} = \frac{{( 7 )}^{2}}{2 ( 32 )} = 0.7656 ft = 9.1875 inches$$

Thus, the maximum height of the jet of solution is 0.7656 feet (9.1875 inches) above the orifice at the end of the 1½ inch diameter discharge pipe.

The cross sectional area of the 1½ inch diameter discharge pipe is given by the following expression.

$$A = π \frac{{( 1 - 1 / 2 )}^{2}}{4}$$

where

A=cross sectional area of the discharge pipe in square inches;

1½=diameter of the discharge pipe in inches;

$${A}_{1} = π \frac{{( 1 - 1 / 2 )}^{2}}{4} = 1.7663 square inches .$$

Therefore, the cross sectional area of the 1½ inch diameter pipe is 1.7663 square inches.

Consider an initial aliquot of aqueous solution which has an arbitrary volume, V₁=0.001 cubic feet=1.728 cubic inches=0.9575 fluid ounces.

The length, L₁, of the initial aliquot of aqueous solution within the 1½ inch diameter discharge pipe is given by the following expression.

$${L}_{1} = \frac{{V}_{1}}{{A}_{1}} = \frac{1.728}{1.7663} = 0.9783 inches = 0.0815 feet$$

The time, Tᵢ₌₁, it takes for the first aliquot (i=1) of aqueous solution to leave the 1½ inch diameter discharge pipe, in seconds, is given by the following expression:

$${T}_{i = 1} = \frac{{L}_{1}}{{v}_{initial}} = \frac{0.0815}{7} = 0.0116 seconds$$

The time it takes for the initial aliquot of aqueous solution to reach maximum height is given by the following expression.

$${T}_{maximum} = \frac{{v}_{initial}}{g} = \frac{7}{32} = 0.2188 seconds$$

In summary, it takes 0.2188 seconds for the first aliquot of aqueous solution to reach the maximum height of 0.7656 feet (9.1875 inches) above the orifice at the end of the 1½ inch discharge pipe yet it takes only 0.0116 seconds for the initial aliquot of solution to be fully discharged from the 1½ inch diameter discharge pipe.

Once the initial jet of aqueous solution reaches its maximum height (i.e., t=0.020+0.0116+0.2188=0.2504 seconds), it spreads out laterally within the 3-inch inside diameter standpipe because it is no longer confined by the inner surface of 1½ inch diameter pipe. As the first aliquot of aqueous solution spreads out to fill the 3-inch diameter standpipe, it assumes a disk-like shape within the 3-inch diameter pipe with a cross sectional area, A₂, given by the following expression.

$${A}_{2} = π \frac{{( 3 )}^{2}}{4} = 7.065 square inches$$

Accordingly, the initial aliquot of aqueous solution assumes the shape of a disk with area equal to 7.065 square inches and thickness, Hᵢ, given by the following expression.

$${H}_{i} = \frac{{V}_{i}}{{A}_{2}} = \frac{1.728}{7.065} = 0.2446 inches$$

Thus, the initial aliquot of aqueous solution occupies the top 0.2446 inches in a growing column of aqueous solution within the 3-inch diameter standpipe.

Each subsequent aliquot of aqueous solution occupies the next lower 0.2446 inches of a growing column of aqueous solution within the 3-inch diameter standpipe.

As later aliquots of aqueous solution enter the 3-inch standpipe, they transfer momentum to the growing column of aqueous solution within the 3-inch diameter standpipe by means of collisions. In effect, the upward jet stream runs into (i.e., collides with) all previous aliquots of aqueous solution, above. The momentum transfer exactly counteracts the effect of gravity on the growing column of aqueous solution in the standpipe. The net effect is that the height of the column of aqueous solution within the 3-inch diameter standpipe remains constant at a height of 0.7656 feet (9.1875 inches) above the end on the 1½ inch discharge pipe.

The length of the suspended column of aqueous solution in the 3-inch diameter standpipe depends upon the length of time the valve is left in the “on” position. For every 0.0116 seconds the on/off valve remains open, the suspended column grows 0.2446 inches, as demonstrated in the calculation, above.

The number, N, of aliquots of aqueous solution required to fill the 3-inch diameter standpipe with 32 fluid ounces of aqueous solution is given by the following expression.

$$N = \frac{32}{( 0.9575 )} = 33.42$$

The maximum length of the column of aqueous solution within the 3-inch diameter standpipe is given by the following expression.

L=(33.42)(0.2446)=8.174 inches=0.6812 feet

The maximum length of the suspended column of aqueous solution is determined by the period of time the valve is left open, the height of the initial upward jet of solution and other geometric factors relating to the diameter of the discharge pipe and the diameter of the standpipe. In the instant case where the diameter of the discharge pipe is 1½ inches and the diameter of the standpipe is 3 inches, the maximum length of the suspended column of aqueous solution is 0.6812 feet (8.174 inches). The top of the column of the aqueous solution is 0.7656 feet above the end on the 1½ inch discharge pipe. The bottom of the column of the aqueous solution is located a distance of 0.6812 feet (8.174 inches) below the top of the column of the aqueous solution.

In other words, filling of the 3-inch diameter standpipe continues until the growing column of aqueous solution is 0.6812 feet (8.174 inches) in length. Filling beyond this level causes a hydrodynamic instability that allows leakage from the bottom of the open-ended 3-inch diameter standpipe. The maximum stable length of the suspended column of aqueous solution is 0.6812 feet (8.174 inches). Accordingly, the bottom of the column of aqueous solution is located at a height of 0.7656−0.6812=0.0844 feet (9.1875−8.174=1.0128 inches) above the orifice of the 1½ inch discharge pipe.

Accordingly, it takes a total duration of time, T_{filling}, to fill the standpipe to the maximum length of 0.6812 feet (8.174 inches). The duration of time required to fill the standpipe, T_{filling}, is given by the following expression.

T_{filling}=(33.42)(0.0116)=0.388 seconds

Since Tₘₐₓᵢₘᵤₘ<T_{filling} (i.e., 0.2188<0.388), the limiting time factor which governs the cycle time is the filling time rather than the time of flight of the first aliquot.

$${T}_{maximum} = \frac{{v}_{initial}}{g} = \frac{7}{32} = 0.2188 seconds$$

When the 3-inch diameter standpipe is filled with 32 fluid ounces of aqueous solution, the on/off irrigation valve is closed, the flow out the 1½ inch discharge pipe is zero. Without continued momentum transfer to support the column of aqueous solution, the aqueous solution falls to the ground out through the bottom open end of the 3-inch diameter standpipe.

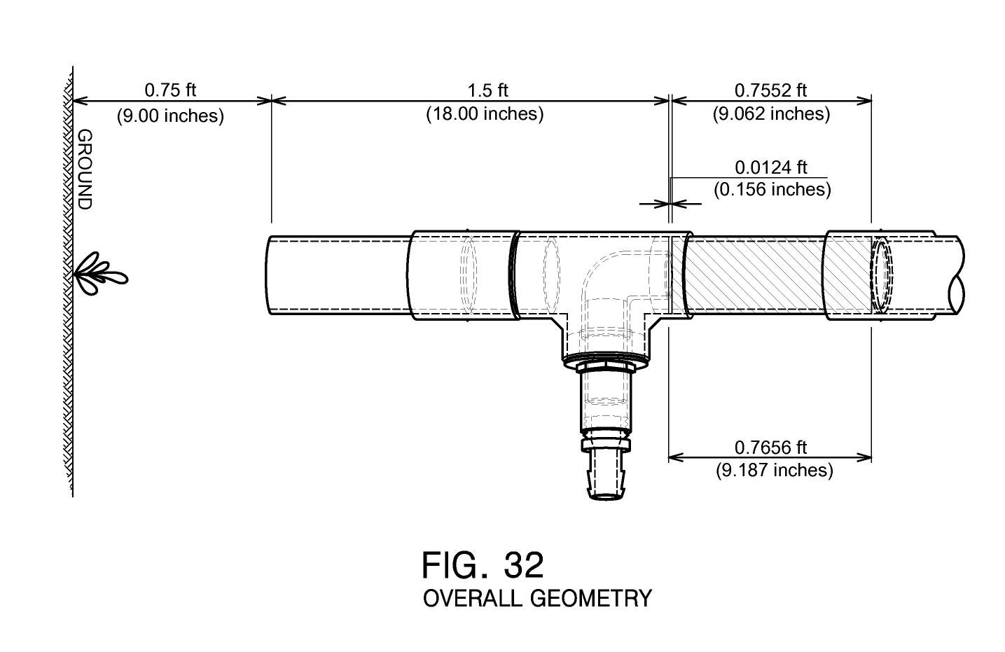

The orifice of the 1½ inch discharge pipe is located a height of 1.5 feet (18 inches) above the bottom of the 3-inch diameter standpipe (see FIG. 32).

While in use, the bottom of the 3-inch diameter standpipe is held 0.75 feet (9 inches) off the ground (see FIG. 32).

Recall that the bottom of the 32-ounce suspended column of aqueous solution is 0.0844 feet (1.0128 inches) above the orifice of the 1½ inch discharge pipe.

In total, the bottom of the suspended column of aqueous solution is 1.5+0.75+0.0844=2.3344 feet above the ground following a slight delay after the on/off irrigation valve is closed. The reason for the slight delay is to allow the “last in” aliquot of aqueous solution to ascend to the bottom of the suspended column of aqueous solution. The time lag is 0.0124 seconds which is the time of flight of the last aliquot from the moment it leaves the discharge pipe until it merges with the column of suspended fluid.

The time lag of 0.0124 seconds is readily calculated by solving the equations of motion for the last aliquot as it moves upward with an initial velocity of 7 feet per second and is subject to downwards acceleration due to gravity, as follows.

d²X(t)/dt²=−g

dX(t)/dt²=−gt+v₀

X(t)=−(½)gt² v₀t+X₀

where

g=32 feet per second squared (gravitational acceleration)

v₀=7 feet per second (initial velocity)

X₀=0 (reference height)

X(t)=−(½) (32) t²+(7) t+0

Solve for the time, t, given X(t)=0.0844 feet, as follows.

0.0844=−(½) (32) t²+(7) t+0

The solution of the equation, above, is t=0.0124 seconds. This is the time of flight of the last aliquot from the moment it leaves the discharge pipe until the moment it merges with the column of suspended aqueous solution.

The height of the last aliquot above the ground, H_{(i=N)}, is given by the following sum.

H_{(i=N)}=m=(0.75)+(1.5)+(0.0124)=2.3344 feet

The time it takes for the lowest (i.e., i=N) aliquot (i.e., “last-in”) of aqueous solution to fall to the ground from a height, H_{(i=N)}, measured from the surface of the ground is given by the following equation.

$${H}_{( i = N )} = \sqrt{\frac{2 {H}_{( i = N )}}{g}} = \sqrt{\frac{2.3344}{16}} = 0.382 seconds$$

The height of the first aliquot above the ground, H₍ᵢ₌₁₎, is given by the following sum.

H₍ᵢ₌₁₎=(0.75)+(1.5)+(0.7656)=3.0156 feet

The time it takes for the highest (i.e., i=1) aliquot (i.e., “first-in”) of aqueous solution to reach the ground is given by the following equation.

$${H}_{( i = 1 )} = \sqrt{\frac{2 {H}_{( i = 1 )}}{g}} = \sqrt{\frac{3.0156}{16}} = 0.434 seconds$$

The on/off irrigation valve is fully closed at time t=0.020+(33.42) (0.0116)+0.020=0.4277 seconds.

The suspended column of solution within the 3-inch diameter standpipe walls begins its free fall towards the ground under the influence of gravity after a time lag of 0.0124 seconds. As explained above, the 0.0124 seconds is the time of flight of the last aliquot from the moment it leaves the discharge pipe until it merges with the column of suspended aqueous solution.

The suspended column of aqueous solution begins its fall at time t=0.020+[(33.42) (0.0116)]+0.020+(0.0124)=0.4401 seconds.

Irrigation commences at time t=0.020+[(33.42) (0.0116)]+0.020+(0.0124)+(0.382)=0.8221 seconds.

Irrigation ends at time t=0.020+[(33.42) (0.0116)]+0.020+(0.0124)+(0.434)=0.8741 seconds.

In summary: (i) at time t=0.4401 seconds, the suspended column of aqueous solution begins to fall; (ii) at time t=0.8221 seconds, the “last-in” aliquot of solution reaches the ground; and (iii) at time t=0.8741 seconds, the “first-in” aliquot of solution reaches the ground. Thus, this is a “last-in/first-out” delivery system.

Irrigation (i.e., the delivery of aqueous solution) takes place over a time interval, τ_{irrigation}, given by the following expression.

T_{irrigation}=(0.8741)−(0.8221)=0.052 seconds

During this period of time, the total volume, V, of solution delivered to the ground is given by the following expression.

V=N Vᵢ=(33.42)(0.001)=0.0334 cubic feet

The time, τ_{irrigation}, required for the delivery of the solution is τ_{irrigation}=0.052 seconds.

The flow rate, F in cubic feet per second for delivery of aqueous from the open-ended 3-inch diameter standpipe is given by the following expression.

$$F = V / ( {τ}_{irrigation} ) = \frac{0.0334}{0.052} = 0.6423 cfs$$

The first aliquot of aqueous solution (i=N) that reaches the ground falls from a height of 2.3344 feet in 0.382 seconds. Therefore, it has an impact velocity, v_{(i=N)}, _{final}, given by the following expression.

v_{(i=N)},_{final}=g(0.382)=12.22 feet per second

The last aliquot of aqueous solution (i=1) that reaches the ground falls from a height of 3.0156 feet in 0.434 seconds. Therefore, it has an impact velocity, v₀=₁), _{final}, given by the following expression.

v₍ᵢ₌₁₎,_{final}=g(0.434)=13.89 feet per second

On average, the aqueous solution falling to the ground has an impact velocity of approximately 13 feet per second.

Thus, the apparatus is capable of delivering 0.0334 cubic feet of aqueous solution with an impact velocity on the order of 13 feet per second. This impact velocity is sufficiently low that vegetative structures are not damaged by this method of irrigation nor is there any appreciable soil erosion. The delivery of 0.0334 cubic feet of aqueous solution occurs over the brief time period of only 0.052 seconds.

Once the standpipe is empty, the working cycle can start all over again.

The time required for one complete working cycle comprises five distinct time periods, as follows.

Time for one complete working cycle={time required to open the irrigation valve}+{time required to deliver the aqueous solution into the standpipe}+{time required to close the irrigation valve}+{time required to assemble the column of aqueous solution}+{time required to empty the standpipe}.

In this example,

0.020 seconds={time required to open the irrigation valve}
0.3877 seconds={time required to deliver the aqueous solution into the standpipe}
0.020 seconds={time required to close the irrigation valve}
0.0124 seconds={time required to assemble the column of aqueous solution}
0.434 seconds={time required to empty the standpipe}

Thus, it takes 0.020+0.3877+0.020++0.0124+0.434=0.8741 seconds to complete one complete working cycle. This is termed the period of the cycle.

Assuming the tractor travels at 3 miles per hour the period of the cycle can be repeated in a path length along a row of crops, L₂, given by the following expression.

L₂=(0.8741)(3)(1.4667)=3.85 feet

[NOTE: 1 mile per hour equals 1.4667 feet per second.]

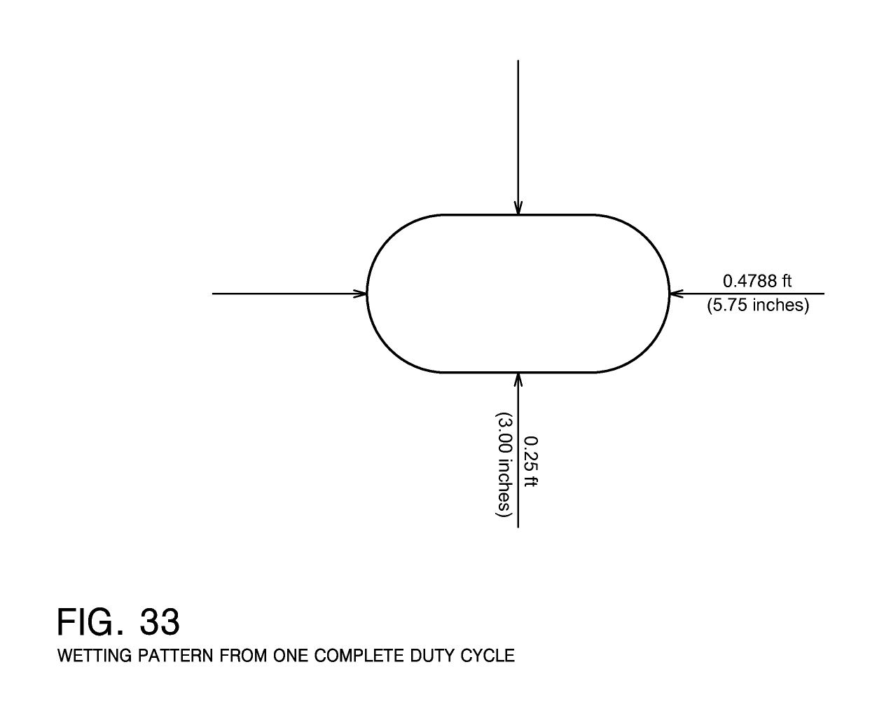

The resulting wetting pattern is the union of two semi-circles plus one rectangle. The major and minor axes are given below (see FIG. 33).

Minor axis=0.25 ft

Major axis=0.25+[(0.052)(3)(1.4667)]=0.478 ft

Such a tight dispersion pattern is desirable because irrigation is not wasted on bare soil as might otherwise be the case.

An exemplary embodiment of the invention provides a method that controls the “on-off” switch that opens and closes the irrigation valve that controls the flow of aqueous solution into the standpipe. In an embodiment, the essential input for the automated control system that triggers the “on-off” switch and marks the onset of the working cycle described above is an optical sensor that detects a plant.

The optical detection of a plant is made possible in agricultural operations by the use of two light (radiation) emitters, powered by an internal power source and modulated to switch on and off at very high speeds. Each emitter emits radiation of a different emitter wavelength. The on/off modulation of one emitter is phase shifted by approximately 90° with respect to the modulation of the other emitter. The pair of emitters is focused on a particular spot on the ground. The light beams, provided by the emitters, are reflected off a plant or the soil and are intercepted by a photo-detector. Because plants have a characteristic spectral reflectance in regions of the electromagnetic spectrum which can be discriminated from the spectral reflectance of the background earth, the relative amplitudes of the reflected radiation at the two emitter wavelengths varies depending on whether the radiation is reflected off a plant or the soil. A ratio of the radiation at the two-emitter wavelengths received by the photo-detector is converted to a phase. This phase is compared to an initial reference phase of the modulation of one of the emitters. A controller uses this phase information to determine the presence or absence of a plant. This digital information can be used to trigger the onset of the working cycle for the irrigation apparatus described above.

The apparatus and method according to this invention is/are capable of operating under a wide variety of conditions, including windy conditions, bright sunlight, artificial illumination, or total darkness, thereby allowing 24 hour operation. Applying fertilizer, herbicide and pesticide at night has significant advantages because cooler conditions allow longer and more effective working hours at critical times during season. Specifically, the higher relative humidity at night aids foliage wetting, thereby prolonging the effectiveness of the herbicide and pesticide, and the absence of wind after sunset eliminates drift. Therefore, in addition to significantly reducing the cost of fertilizer, herbicide and pesticide associated with the localized irrigation of plants, the present invention, by having the capability of operating at night, provides additional advantages.

The use of an optical control apparatus has not been practical in controlling an irrigation apparatus in the prior art because the output from the optical control apparatus is low voltage and low amperage electrical power. This invention provides for the use of timers and load relays to boost the voltage and amperage of the electrical power so as to be available to operate heavy-duty irrigation valves.

In summary, the use of a precision hydraulic applicator (i.e., standpipe) for the application of aqueous solution in accordance with the present invention ensures that no plant is overlooked, no plant gets more water, fertilizer, herbicide or pesticide than is required, and no material is wasted on bare ground. As a result, the apparatus and method reduce labor, reduce equipment-operating costs, reduce fertilizer and herbicide and pesticide costs, significantly improve control, and dramatically reduce exposure to the fertilizer, herbicide and pesticide of both the crop and the workers.

### DETAILED DESCRIPTION OF THE DRAWINGS

The following description is meant to be illustrative only and not limiting. Other embodiments of this invention may also apply in view of this disclosure.

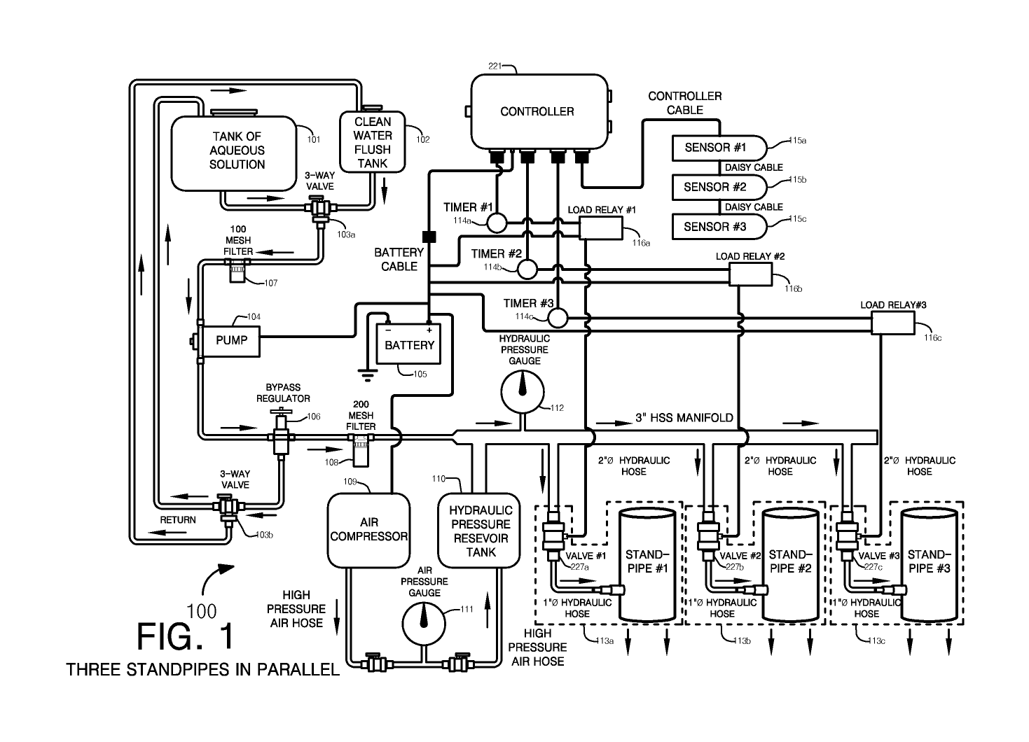

FIG. 1 is an embodiment of the apparatus and electronic controls and method for delivering a predetermined quantity of aqueous solution to delicate plants in a localized fashion and by means of delivering discrete aliquots of low velocity aqueous solution.

In an exemplary embodiment, the apparatus and method according to the invention are illustrated in (i) a plumbing schematic diagram (FIG. 1); and (ii) the following sequence of events.

Step 1—In an embodiment, a plurality of sensors (WeedSeeker® Model 650) 115 detects a plant and send that information to the master controller (Model NTech 151) 221 by means of a daisy chain cable (Model 400-1-029) by means of electronic signals.

Step 2—In an embodiment, the master controller (NTech Model 151) 221 sends electronic signals to three solid-state timers (Model OMRON H3CA) 114a, 114b and 114c by means of a daisy chain cable (Model 400-1-029).

Step 3—In an embodiment, following a programmable time delay the solid-state timers (Model OMRON H3CA) 114a, 114b and 114c send electronic signals to external solenoid valves (Model GOYEN 3QH-5N-V) 227a, 227b and 227c, respectively.

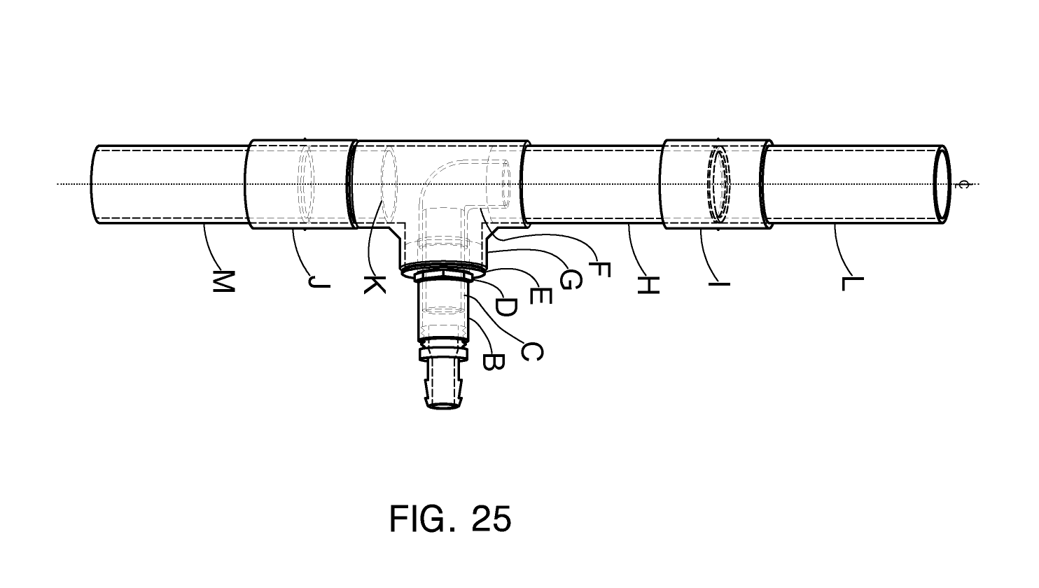

Step 4—In an embodiment, once the external solenoid valves (Model GOYEN 3QH-5N-V) 227a, 227b and 227c have been activated, aqueous solution flows under high pressure (ranging from 100 psi to 60 psi) through a flexible hose into the hydraulic standpipe assembly ABCDEFGHIJKLM (see FIG. 25).

In summary, the electronic control system comprises an internal dual-wavelength light source, an optical detector and optics module, and an electronics module. The operation of the sensors is based on the fact that every substance has a unique spectral reflectance signature. The sensors are optimized to compare the reflected light from soils and plants. The presence of chlorophyll in plants results in a distinct reflectance signature at particular wavelengths as compared to bare soil. The sensors compare the reflected light at two different wavelengths. When a plant comes into the field of view, the circuitry “recognizes” the chlorophyll signature and activates the solenoid valve thereby filling the standpipe.

FIG. 1 is an embodiment of a plumbing schematic diagram illustrating that both the tank of aqueous solution 101 and a clean water flush tank 102 are plumbed to a 3-way valve 103a by means of flexible hoses. The effluent from the 3-way valve 103a may be either: (i) aqueous solution from the tank 101 that contains water plus fertilizer, pesticide, or herbicide, or any combination thereof; or (ii) clean water used to flush the hoses, depending upon the orientation of the 3-way valve 103a. The effluent from the 3-way valve 103a is filtered by means of a #100 mesh filter 107 and comprises the intake for a high capacity hydraulic driven centrifugal pump (Model FMC-200-HYD-210) 104. The pump 104 is powered by means of a battery 105, or may be powered by the hydraulics system of the tractor that tows the apparatus (not shown). The high pressure (100 psi to 60 psi) effluent of pump 104 is directed to a bypass regulator 106 which is used to direct the effluent towards the #200 mesh filter 108 or to redirect the flow back towards the tank of aqueous solution 101 or to the clean water flush tank 102, depending on the orientation of the 3-way valve 103b. The effluent of the #200 mesh filter 108 flows into a 3 inch hollow structural steel tube (hereinafter called HSS) manifold 113. The 3 inch HSS manifold 113 is connected to a hydraulic pressure reservoir tank 110 that serves to maintain constant pressure. The pressure in the hydraulic pressure reservoir tank 110 is maintained at a constant pressure by means of an air compressor 109. The pressure in the hydraulic pressure reservoir tank 110 is monitored by means of an air pressure gauge 111. The air compressor 109 is powered by means of a battery 105. The pressure 3 inch HSS manifold 113 is in the range of 100 psi to 60 psi, depending on the output of the air compressor. The 3 inch HSS manifold 113 provides a high-pressure (100 psi to 60 psi) supply of aqueous solution to each of three control valves (Model 145H DirectoValve) 227a, 227b and 227c. The control valves (Model 145H DirectoValve) 227a, 227b and 227c are turned “on” and “off” by the master controller (Model NTech 151) 221. The control valves (Model 145H DirectoValve) 227a, 227b and 227c are powered by means of a battery 105 (not shown). The effluent from the (Model 145H DirectoValve) control valves 227a, 227b and 227c provide discrete aliquots of aqueous solution into the three standpipes, #1, #2 and #3 respectively 113a, 113b and 113c. The controller 221, together with the timers 114a, 114b and 114c and the sensors 115a, 115b and 115c and load relays 116a, 116b and 116c, control the flow of aqueous solution into the three standpipes, #1, #2 and #3, respectively. The volume of the aliquots of aqueous solution and their precise timing are determined by the controller 221 in response to plants detected in the visual field of the sensors 115a, 115b and 115c.

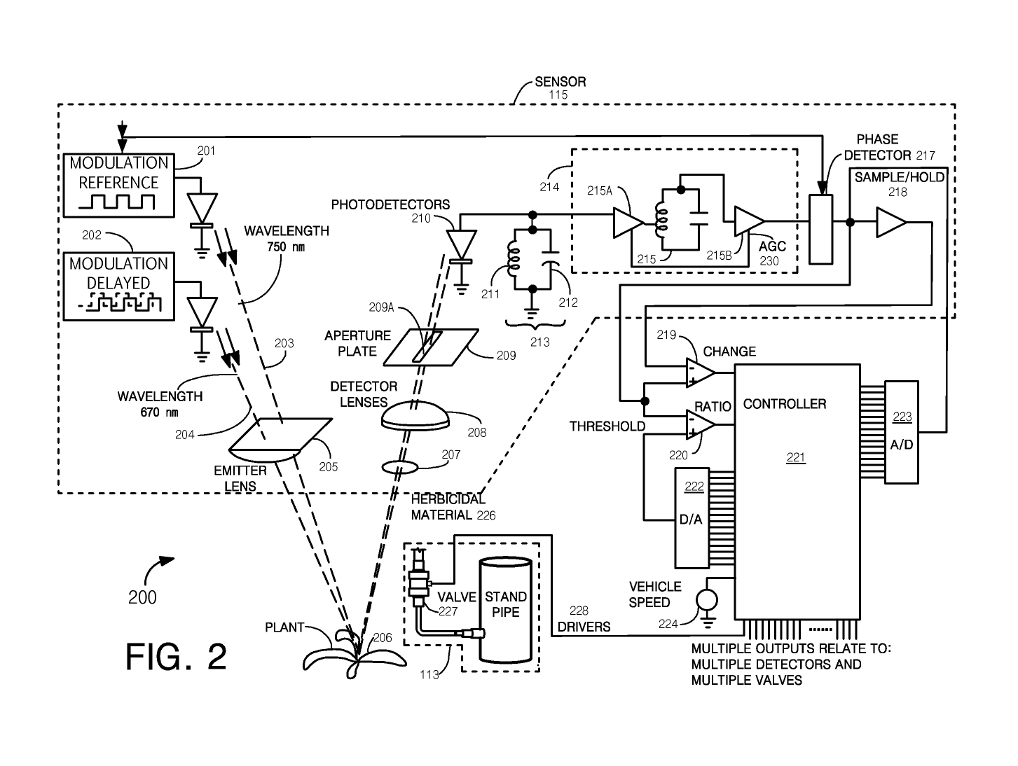

FIG. 2 illustrates one embodiment of an on/off photo-detector type of electronic controller used to regulate the on/off flow of aqueous solution into the apparatus according to the invention. In this embodiment, two monochromatic light sources 201 and 202 are provided.

Monochromatic light source 201 emits a light beam 203 having a wavelength of 750 nanometers, while monochromatic light source 202 emits a light beam 204 having a wavelength of approximately 670 nanometers. Light sources 201 and 202 are typically commercially available light emitting diodes (LEDs) formed from gallium arsenide, gallium arsenide phosphide, or gallium aluminum arsenide, which provide an extremely reliable and cost-effective source of monochromatic light. In another embodiment, other light sources may include lasers or broadband light sources with filters.

In an embodiment, if diodes 201 and 202 are merely turned on with a DC current, and directed at a predetermined surface area 206, the reflection of the sun from surface 206 would significantly impair if not render inoperable structure 200. Thus, in exemplary embodiment, the current to diodes 201 and 202 is selectively modulated. In one embodiment, the modulation is such that diodes 201 and 202 are driven at the highest possible frequency within their bandwidth constraints while remaining compatible with the other components of the system (described below in detail). In this embodiment, diodes 201 and 202 are modulated at a frequency of 455 KHz. Note that as the frequency increases, the more information is available to the user within one predetermined time cycle, thereby providing more sensitive measurements.

In an embodiment, light beams 203 and 204, emitted from diodes 201 and 202, respectively, are focused by emitter lens 205 on a predetermined area of surface 206 which may contain a plant, soil, or a combination of plant and soil. In one embodiment, light beams 207, reflected off surface 206, are detected by photo-detector 210 after passing through detector lens 208 and aperture 209A of aperture plate 209. The light detected by photo-detector 210 comprises different ratios of wavelengths of monochromatic light depending upon whether light beams 203 and 204 are reflected by a plant or by soil.

To ensure proper alignment between detector lens 208, aperture 209A of aperture plate 209, and photo-detector 210, one embodiment of the present invention provides an aperture 209A that is much smaller than detector lens 208. Moreover, this embodiment provides an image of reflected beams 207 through detector lens 208 that is smaller than the size of photo-detector 210. In this manner, any misalignment between these three elements is rendered non-critical so long as the misalignment is within certain limits set by the size of aperture 209A, the size of diode 201 and the distance of diode 201 from aperture 209A, among other parameters. Note that increasing the size of photo-detector 210 produces an undesirable increase in capacitance. In an embodiment, photo-detector 210 is only slightly larger than aperture 209A to minimize capacitance. In another embodiment, this capacitance is buffered by a cascade amplifier circuit, thereby providing substantially the same bandwidth as if a lower capacitance device (i.e. smaller photo-detector 210) were used.

In an embodiment, a tuned circuit 213 comprising inductor 211 and capacitor 212, placed in series with photo-detector 210, resonates due to excitation from photo-detector 210. The output waveform of tuned circuit 213, therefore, remains substantially sinusoidal. Tuned circuit 213 rejects everything which is not sinusoidal and not at a selected frequency (i.e. undesirable harmonics). The accepted sinusoidal waveform is then provided to circuitry 214 that typically comprises amplifiers 215A and 215B and tuned circuit 215.

Photo-detector 210 converts photo-energy from reflected light beams 207 into low-level electrical signals that represent the color signature of the object(s) in the field of view. In an embodiment, a tuned circuit 213 comprising inductor 211 and capacitor 212, placed in series with photo-detector 210, resonates due to excitation from photo-detector 210. The output waveform of tuned circuit 213, therefore, remains substantially sinusoidal. Tuned circuit 213 rejects everything which is not sinusoidal and not at a selected frequency (i.e. undesirable harmonics). The accepted sinusoidal waveform is then provided to circuitry 214 that typically comprises amplifiers 215A and 215B and tuned circuit 215.

In an embodiment, circuitry 214 provides a very high gain for the modulated signals provided by reflected light beams 207 and at the same time the inductor 211 in tuned circuit 213 passes the unwanted DC signal output from diode 210 due to the sunlight directly to ground. Automatic frequency control (AFC) (not shown) is used to ensure that tuned circuit 215 provides the maximum out-of-band rejection, thereby minimizing the interference of sunlight reflected off surface 206 with the modulated light beams 207 reflected from the same surface. In an embodiment, automatic gain control (AGC) 230 is used to provide the widest possible dynamic range for amplifiers 215A and 215B. In another embodiment, circuitry 215 clips the amplified sinusoidal waveform to provide a predominantly squared waveform to phase detector 217. Phase detector 217 takes the squared output waveforms of circuitry 214, and multiplies that waveform by the waveform of diode 201. In this embodiment, phase detector 217 determines the phase shift of the output waveform of circuitry 214 with respect to the original phase of the waveform provided by diode 201. In one embodiment, phase detector 217 is an LM3089 FM receiver IF system manufactured by National Semiconductor.

In an embodiment, to enhance the ability of system 200 to process information rapidly, a sample and hold circuit 218 is coupled to phase detector 217. The output signal from the sample and hold circuit 218 is provided to the negative input terminal of comparator 219. The input signal to the sample and hold circuit 218 is provided to the positive input terminal of comparator 219, to the negative input terminal of comparator 220 and to the analog to digital converter 223. Comparator 219, therefore, provides a comparison of the instantaneous value of the input to sample and hold circuit 218 to the last value detected by sample and hold circuit 218, thus, providing an indication as to whether the instantaneous change in the analog signal being detected by diode 201 is positive or negative. The output signal from comparator 219, provided to controller 221, thus, indicates the direction of change of any particular color signature. Accordingly, comparator 219 supplies information to controller 221 that allows controller 221 to determine the magnitude and direction of the phase shift of the summed reflected radiation with respect to the initial modulated beams 203 and 204.

In an embodiment, ratio comparator 220 compares the output signal of digital to analog converter 222 to the instantaneous value of the output signal from phase detector 217. The threshold reference voltage in one embodiment is adjusted manually for various types of background mineral soil and partially decomposed organic material. In another embodiment, the threshold reference voltage is adjusted automatically by appropriate software in controller 221 and digital-to-analog converter 222. In this manner, controller 221 provides a continuous update of the background material to comparator 220 via digital to analog converter 222. Ratio comparator 220 has a binary output signal, which depends on whether the ratio of wavelengths detected in the field of view exceeds that of the reference background. In an embodiment, controller 221 analyzes signals provided by comparators 219 and 220 and determines whether a weed is detected with the predetermined area of surface 206. Note that although only single components (for example photo-detector 210) are illustrated, system 200 in one or embodiments, comprises a plurality of photo-detectors 210 and the appropriate signal processing circuitry.

In an embodiment, if a plant is detected, controller 221 activates a device to turn on a flow of aqueous solution to the standpipe apparatus 113. As illustrated in FIG. 2, controller 221 provides a drive signal (typically high for transistor 228 as shown) to the base of NPN bipolar transistor 228 if a plant is detected, thereby turning on transistor 228. Turning on transistor 228 subsequently opens a solenoid-actuated valve 227 that releases a jet of aqueous solution into the standpipe (FIG. 26).

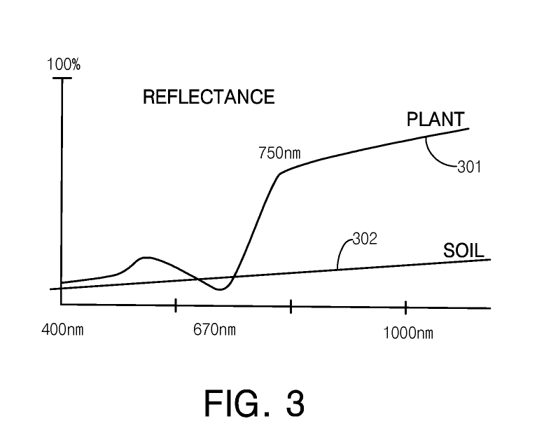

In an embodiment, FIG. 3 illustrates a graph having an x-axis representing wavelength of light from 400 nanometers to approximately 1000 nanometers and a y-axis representing percentage of reflectance. Radiation is reflected from or conversely absorbed by surfaces, depending upon the characteristics of those surfaces. In the case of plants, radiation in the blue and red wavelengths (i.e. 380 nanometers to 700 nanometers) is strongly absorbed by the chlorophylls in the plants while the near infrared wavelength (i.e. 700-1000 nanometers) is strongly reflected. In one or more embodiments, other substances, such as soil, absorb much more of the radiation in many of those wavelengths.

In an exemplary embodiment, FIG. 3 illustrates that for a wavelength of, for example 750 nanometers, a typical plant (represented by curve 301) is easily distinguished from typical soil (represented by curve 302) because the plant reflects a higher percentage of incident light than the soil. Note that a plant has minimum reflectance and, in fact, reflects less than soil at a wavelength of approximately 670 nanometers. In an embodiment, two monochromatic light sources are used to create the reflected light, rather than natural sunlight or an artificial white light source. The optimum light sources to distinguish a plant from soil, as shown in FIG. 2, have a wavelength of approximately 670 nanometers (in the upper-red waveband) and a wavelength of approximately 750 nanometers (in the near-infrared waveband).

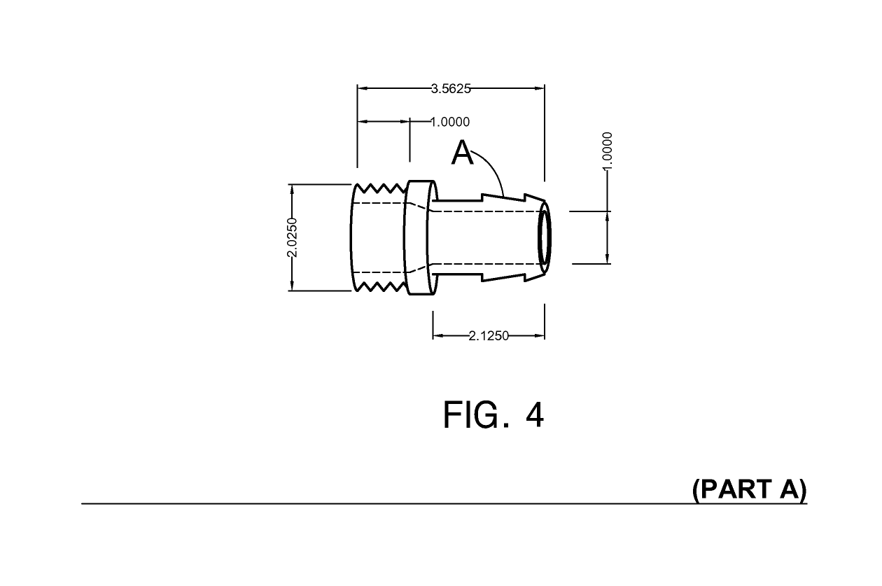

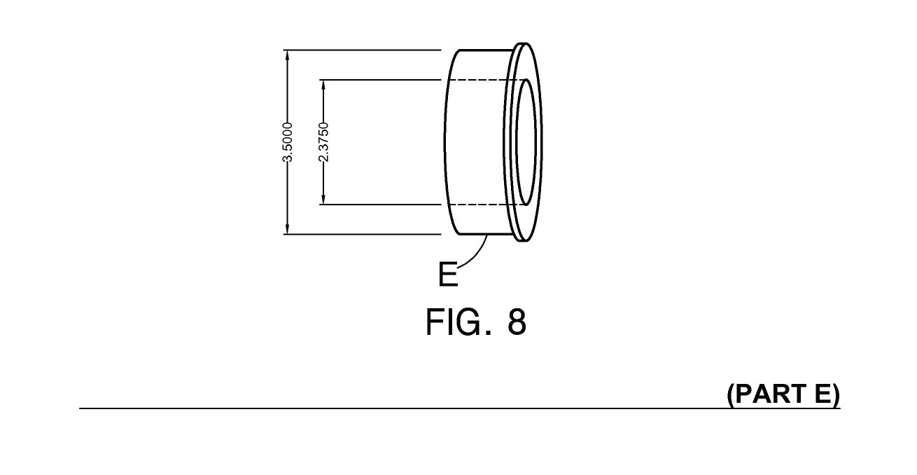

In one embodiment, FIG. 4 through FIG. 26, taken together, illustrate the assembly and use of the hydraulic standpipe, as described in the following sequence.

Step 1: The flow of aqueous solution is delivered to the hydraulic standpipe Assembly ABCDEFGHIJKLMN (FIG. 25) by means of a flexible hose of 1¼ inch (NPS) diameter; the flexible hose connects to the hydraulic standpipe Assembly ABCDEFGHIJKLMN at the barbed fitting Part A (FIG. 4).

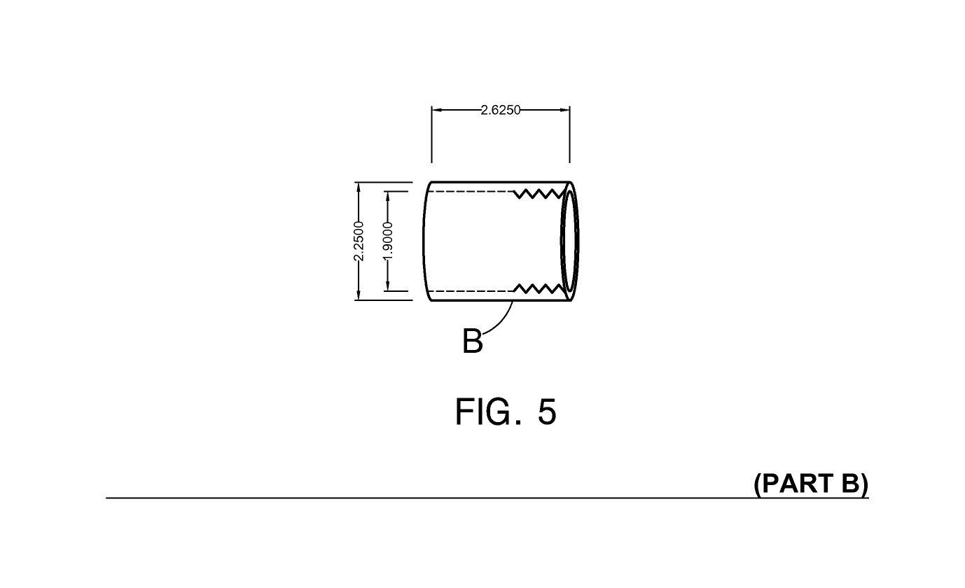

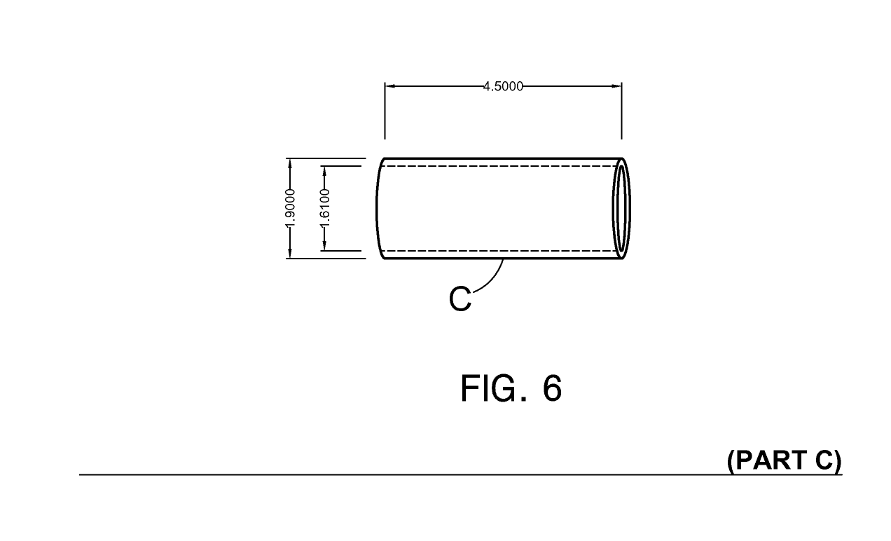

Step 2: The barbed-to-threaded pipe adapter Part A is fastened into the 1½ inch female threaded pipe Part B (FIG. 5). A 1½ inch (NPS) PVC pipe Part C (FIG. 6) is housed within the 1-½ inch female threaded pipe Part B in such a way that the outside diameter of the pipe Part C is flush with the inside diameter of the pipe Part B.

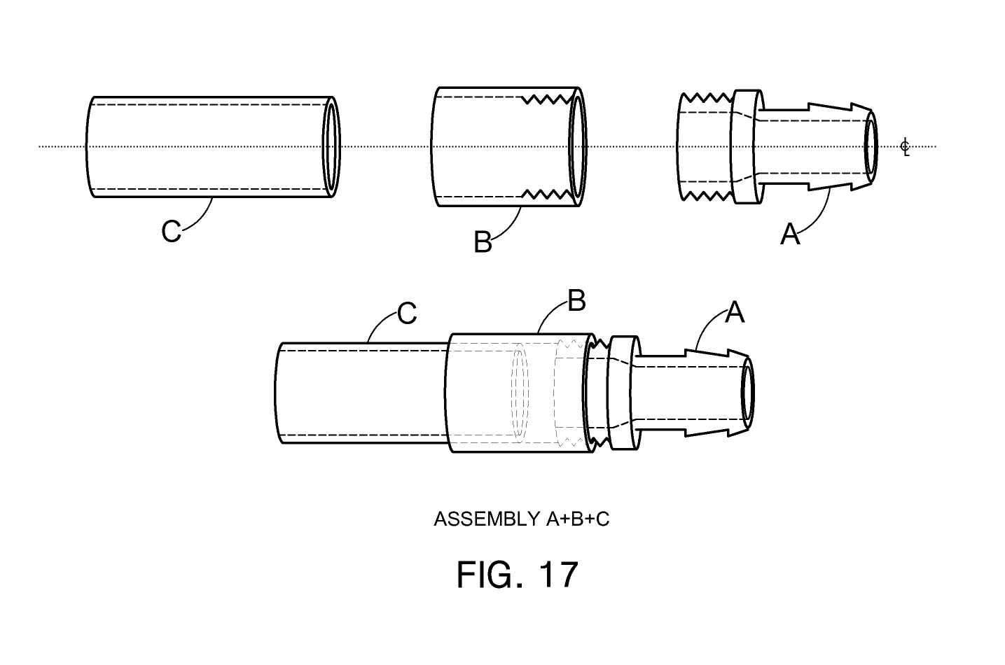

Step 3: The connection between Parts A, B and C creates an assembly of Parts Assembly ABC (FIG. 17).

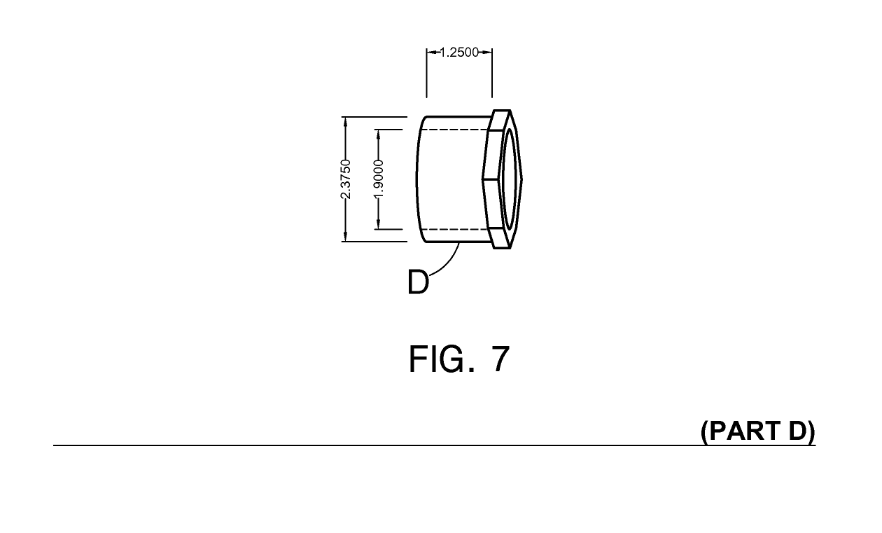

Step 4: One end of the pipe Part C is flush to the threaded end of the pipe adapter Part A so as to retain a contiguous inner diameter among the pipe adapter Part A and pipe Part C. The other end of the pipe Part C extends beyond the edge of the pipe Part B where it enters a 1½ inch-to-2 inch pipe increaser Part D (FIG. 7).

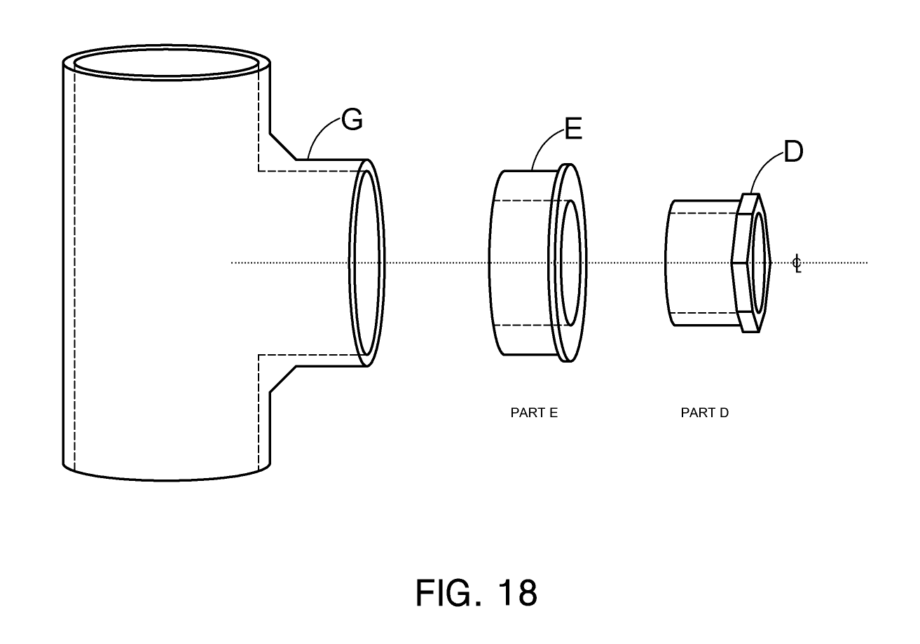

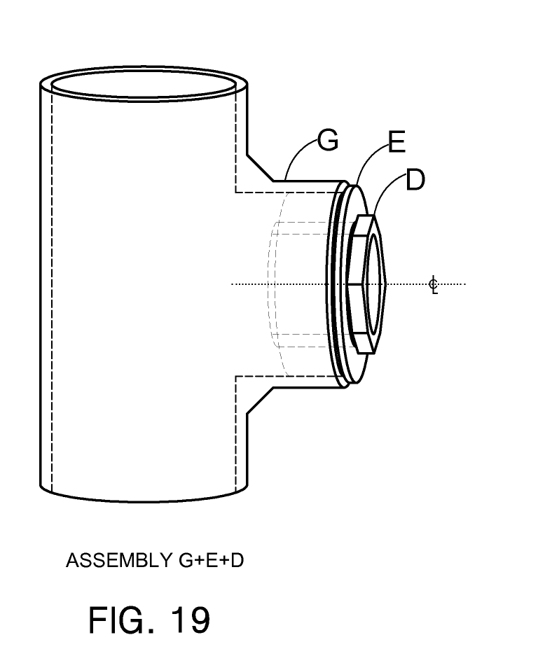

Step 5: The pipe increaser Part D is housed within a 2 inch-to-3 inch pipe increaser Part E in such a way that the outside diameter of the pipe increaser Part D is flush with the inside diameter of the pipe increaser Part E see FIGS. 18 and 19.

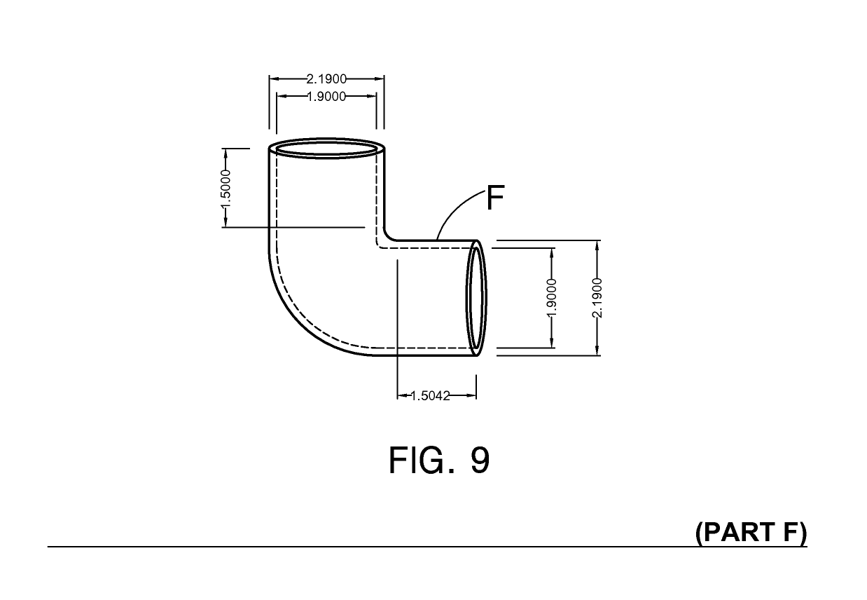

Step 6: The pipe Part C extends beyond the edge of both the pipe increaser Part D and Part E where the end is fitted with a 1½ inch pipe elbow of 90 degrees Part F (FIG. 9).

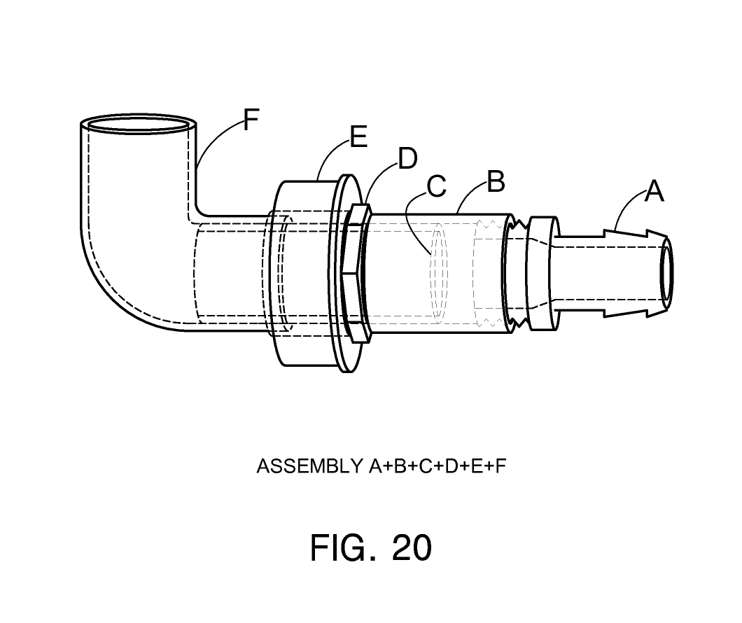

Step 7: The connection between Parts A, B, C, D, E and F creates Assembly ABCDEF (FIG. 20).

Step 8: The pipe Part C extends beyond the edge of both the pipe increaser Part D and Part E where the end is fitted with a 1½ inch pipe elbow of 90 degrees Part F (FIG. 9); the composite of Parts A, B, C, D, E and F creates Assembly ABCDEF (FIG. 20).

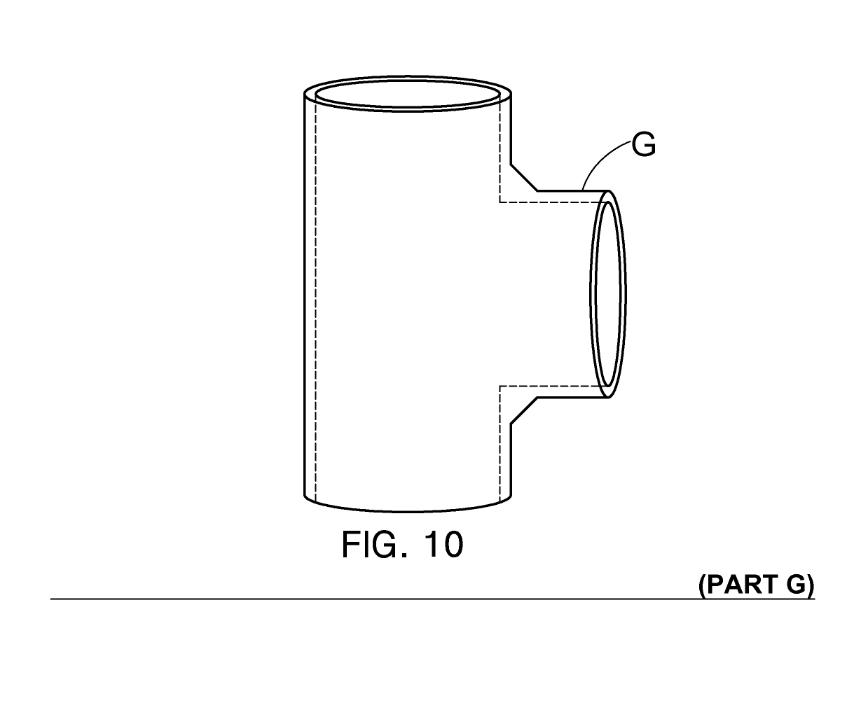

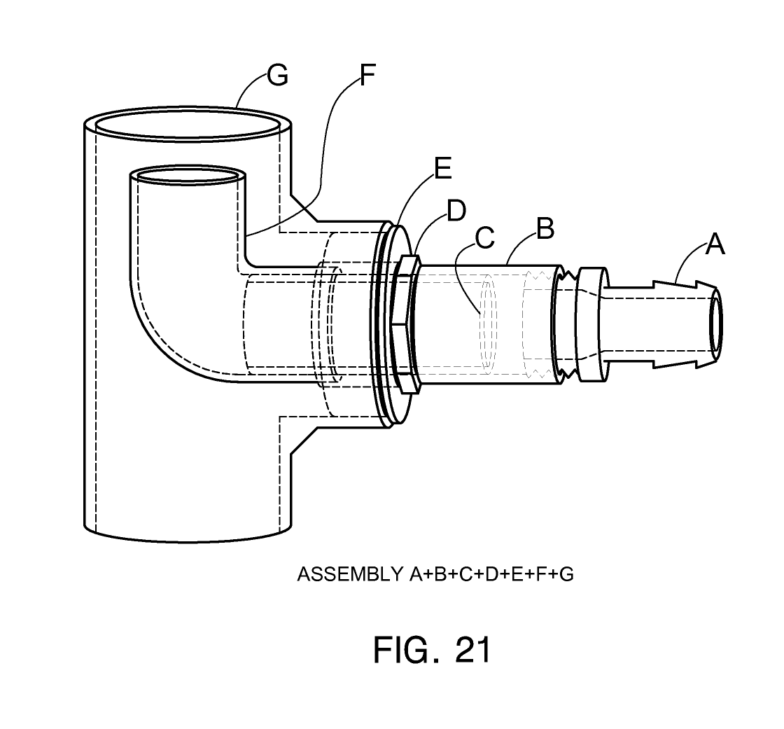

Step 9: Assembly ABCDEF is fastened to a 3 inch (NPS) pipe tee Part G (FIG. 10) at the opening perpendicular to the transverse openings in such a way that the pipe elbow Part F is coaxially aligned with one of the transverse opening of the pipe tee Part G; the composite of Part G and Assembly ABCDEF creates Assembly ABCDEFG (FIG. 21).

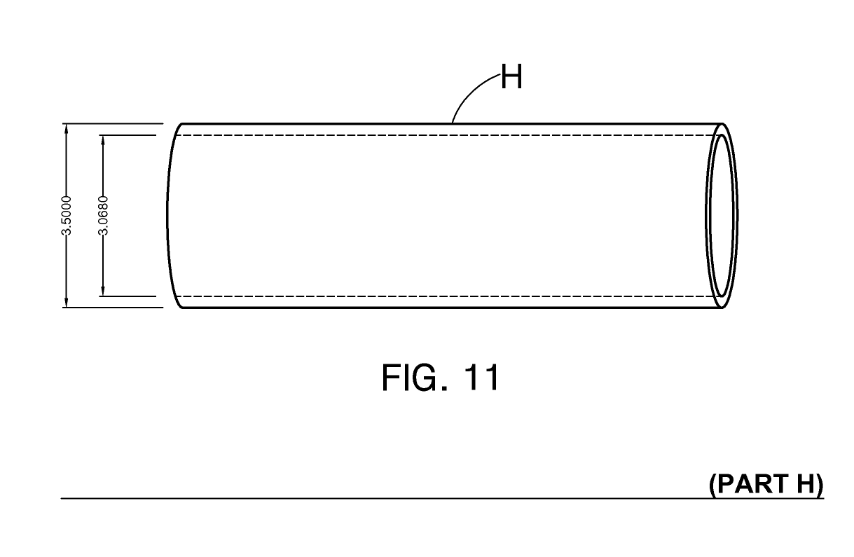

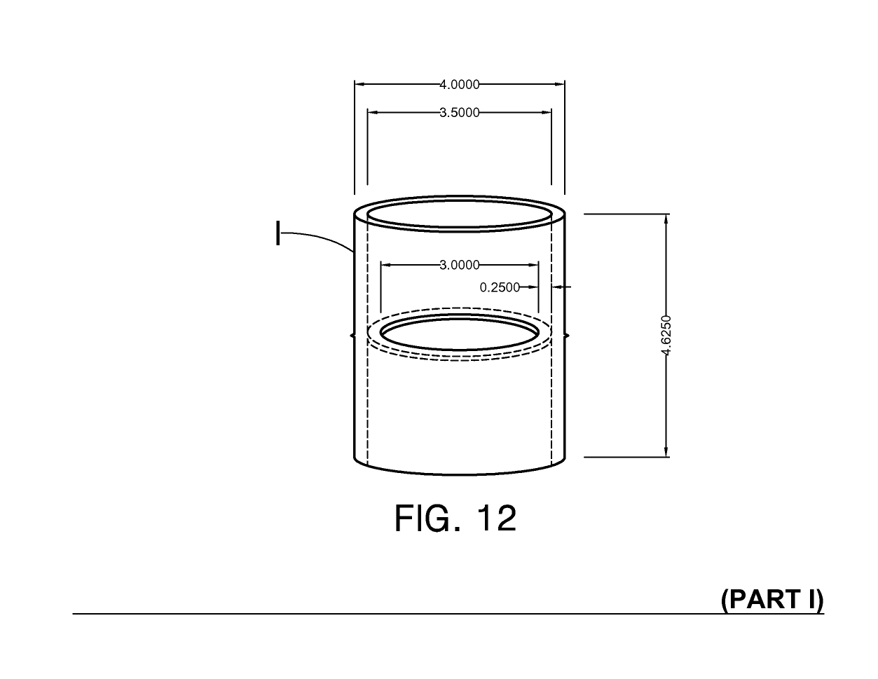

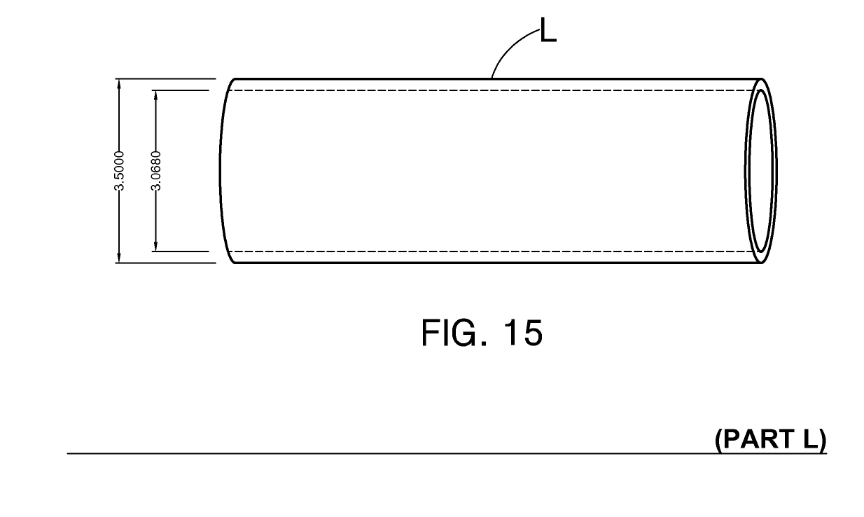

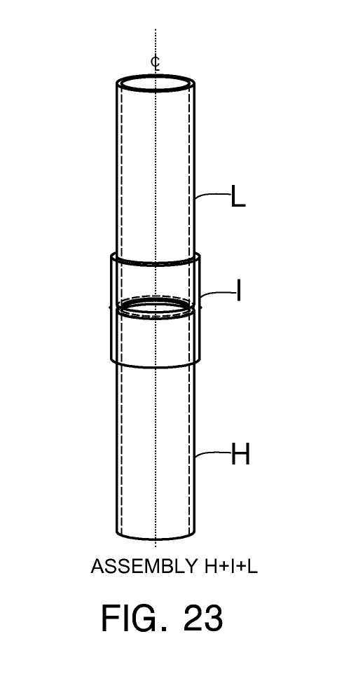

Step 10: The 3 inch (NPS) pipe Part H (FIG. 11) is connected to a 3 inch (NPS) pipe Part L (FIG. 15) by means of a 3 inch pipe coupling Part I (FIG. 12); the composite of Parts H, L and I create Assembly HIL (FIG. 23).

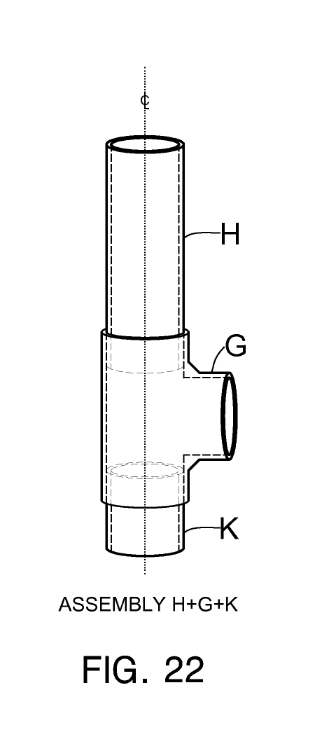

Step 11: The open end of Assembly HIL located at the pipe Part H is attached to the pipe tee Part G (see FIG. 22) located above the coaxially aligned pipe elbow Part F (see FIG. 21).

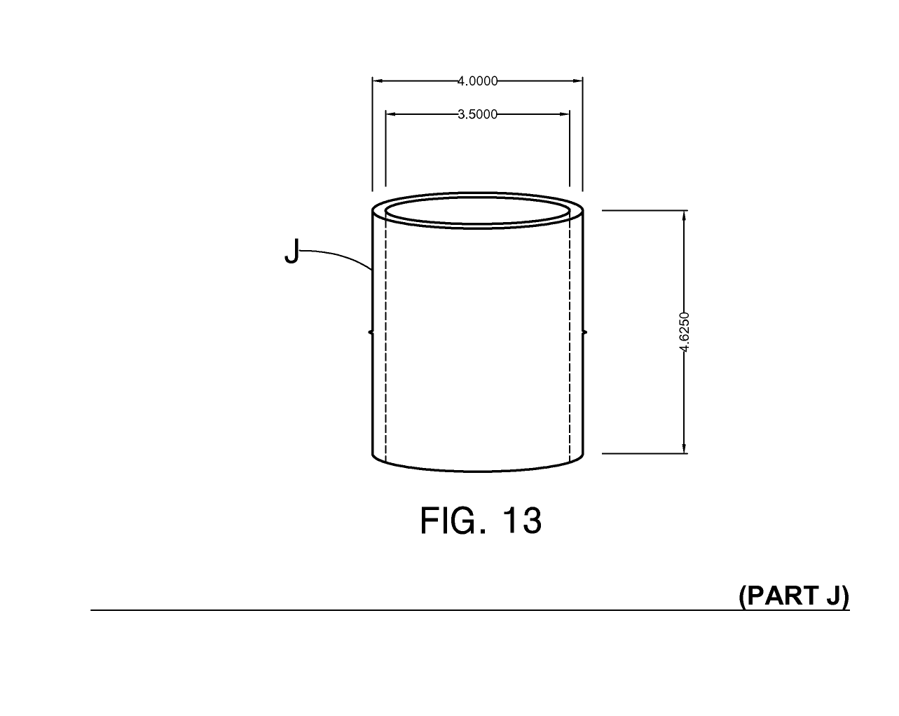

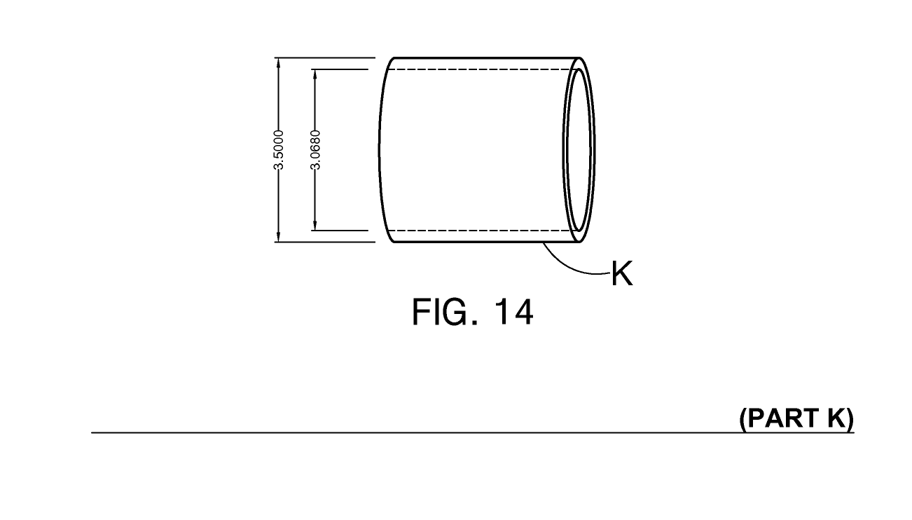

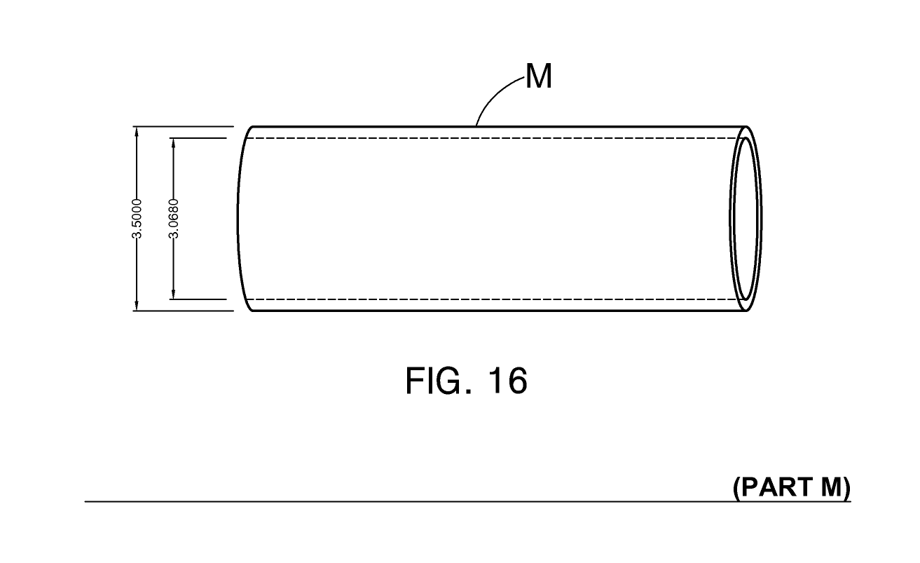

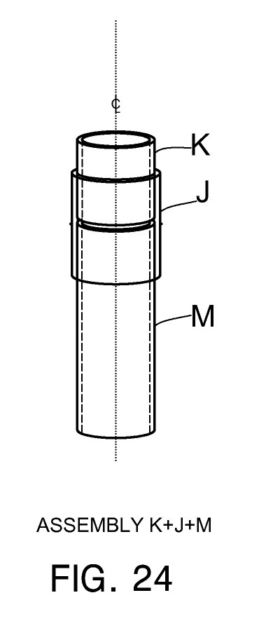

Step 12: The 3 inch (NPS) pipe Part M (FIG. 16) is connected to a 3 inch (NPS) pipe Part K (FIG. 14) by means of a 3 inch pipe coupling Part J (FIG. 13); the composite of Parts K, J and M creates Assembly KJM (FIG. 24).

Step 13: The open end of Assembly KJM located at the pipe Part K is attached to the pipe tee Part G (see FIG. 22) located below the coaxially aligned pipe elbow Part F (see FIG. 21).

Step 14: The composite of Assembly ABCDEFG, Assembly HIL and Assembly KJM creates the hydraulic standpipe Assembly ABCDEFGHIJKLM see FIGS. 25 and 26.

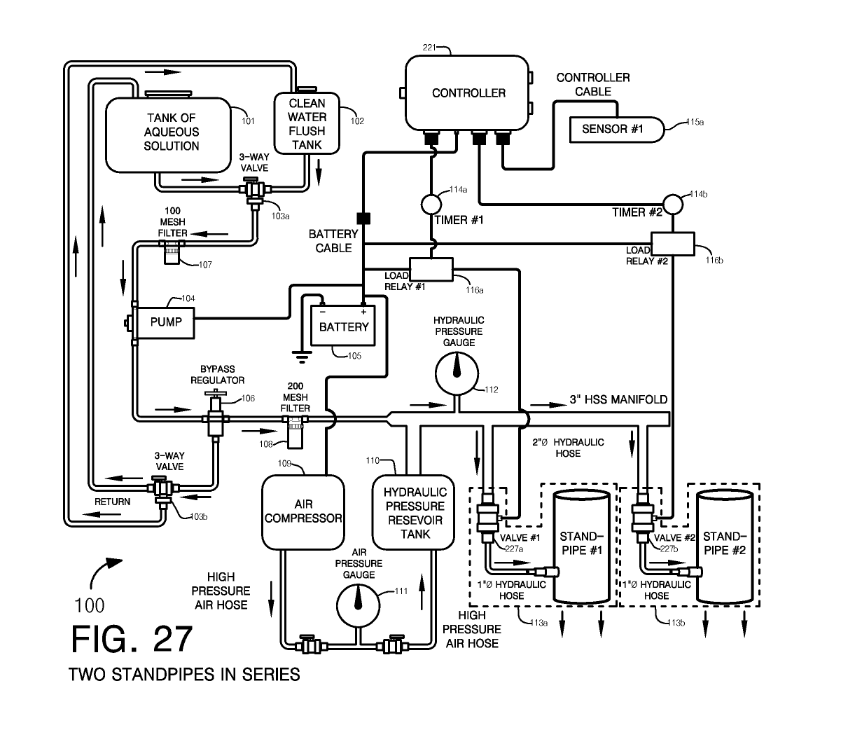

In an embodiment, FIG. 27 illustrates the overall schematic where two standpipes are used in series. In this embodiment, the timers are wired in parallel.

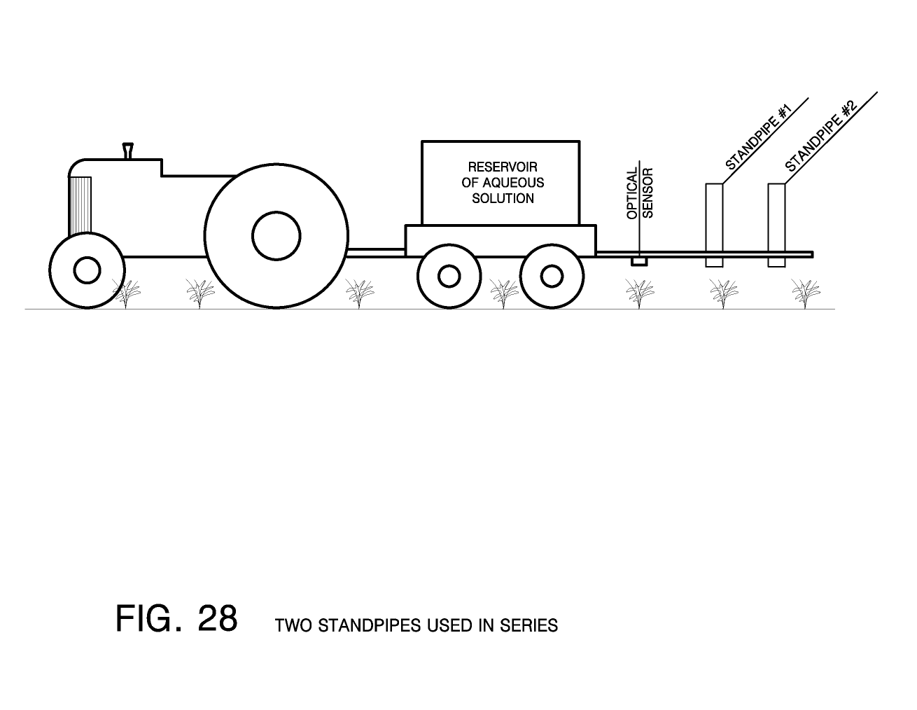

In an embodiment FIG. 28 illustrates the use of two standpipes used in parallel. In this embodiment the entire assembly is towed behind a tractor.

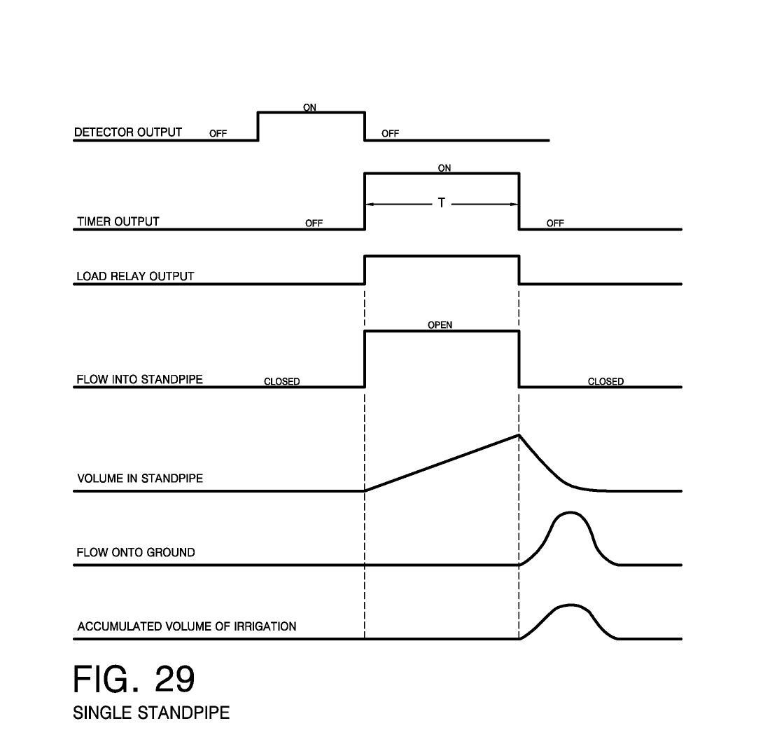

In an embodiment, FIG. 29 illustrates the timeline of the sequence of events starting with the detection of a plant by optical means through one complete duty cycle using the apparatus according to the invention.

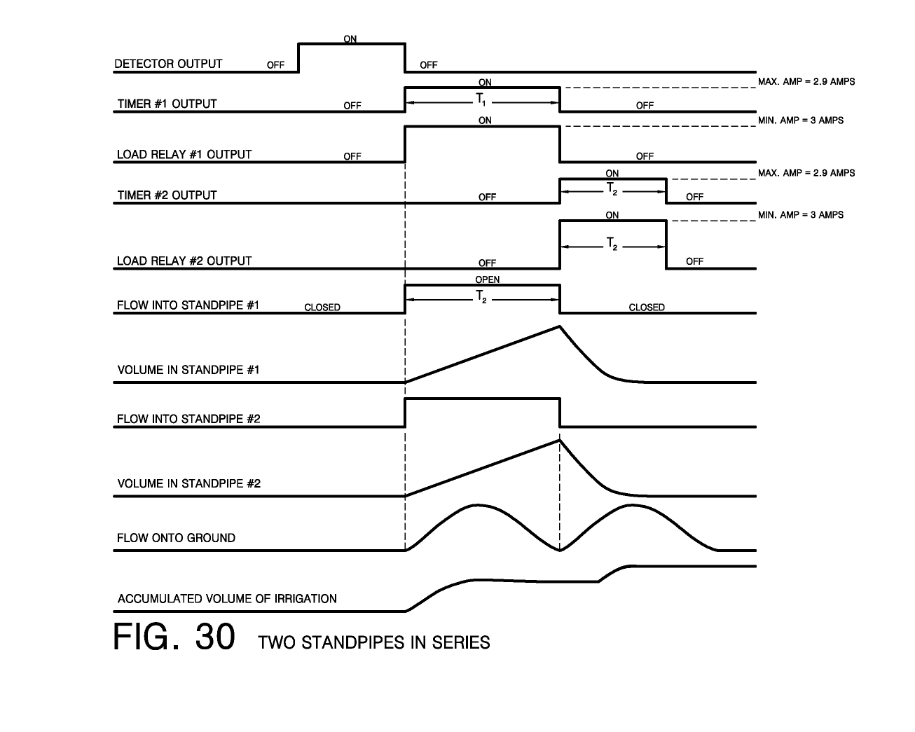

In an embodiment, FIG. 30 illustrates the timeline of the sequence of events (as FIG. 29, above) where two standpipes are used in series.

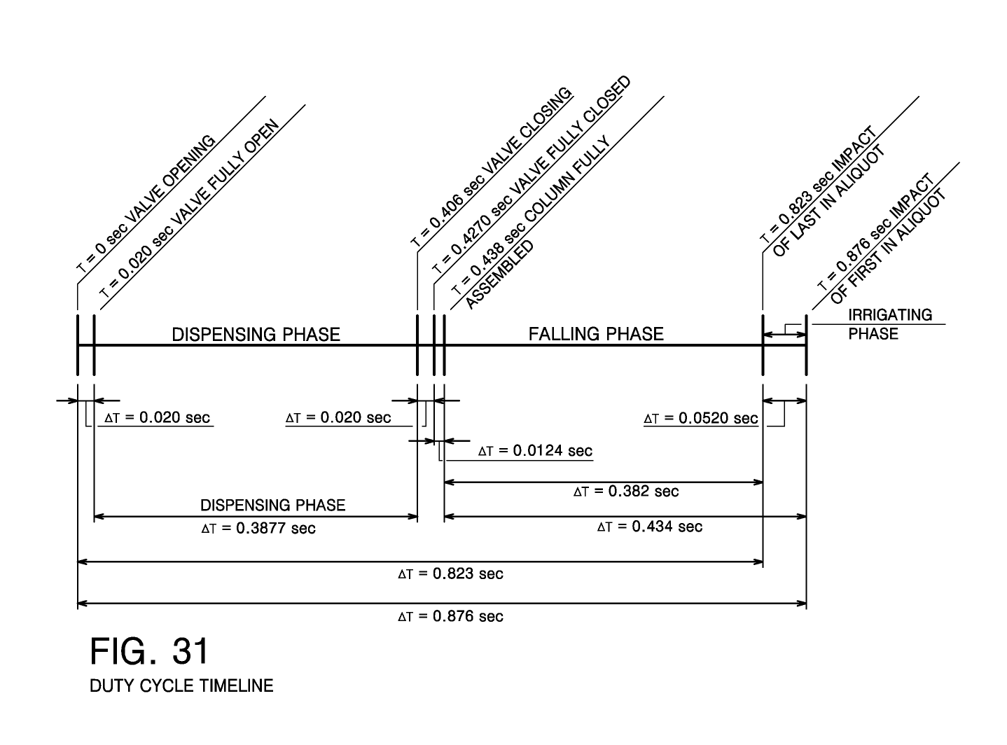

In an embodiment, FIG. 31 illustrates the timeline of one complete duty cycle.

In an embodiment, FIG. 32 illustrates the overall geometry of the standpipe in use.

In an embodiment, FIG. 33 illustrates the spatial pattern of wetting upon the ground as a result of one complete duty cycle.

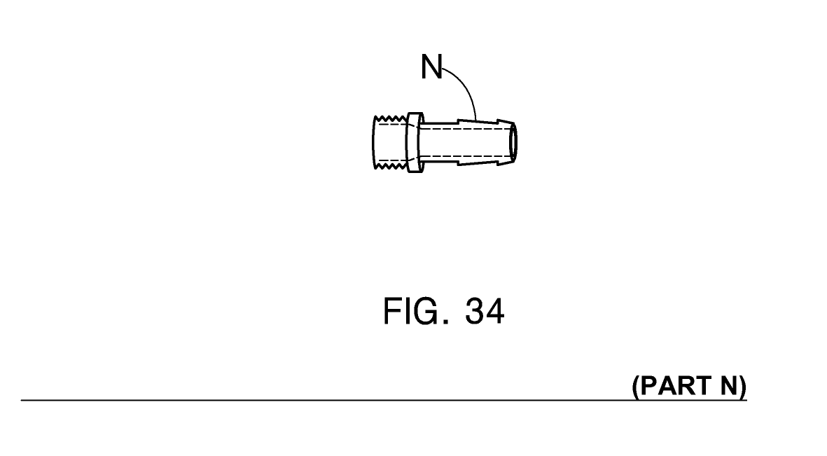

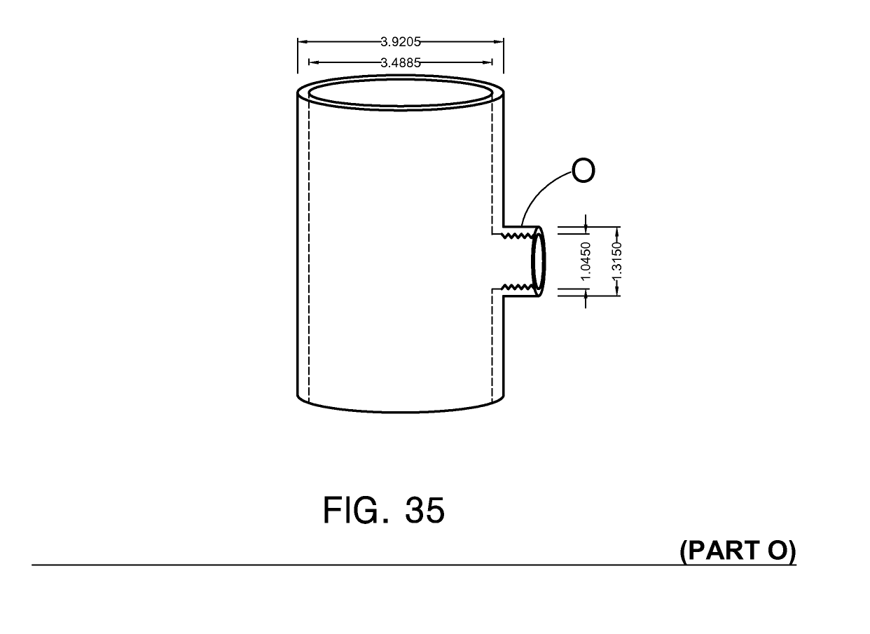

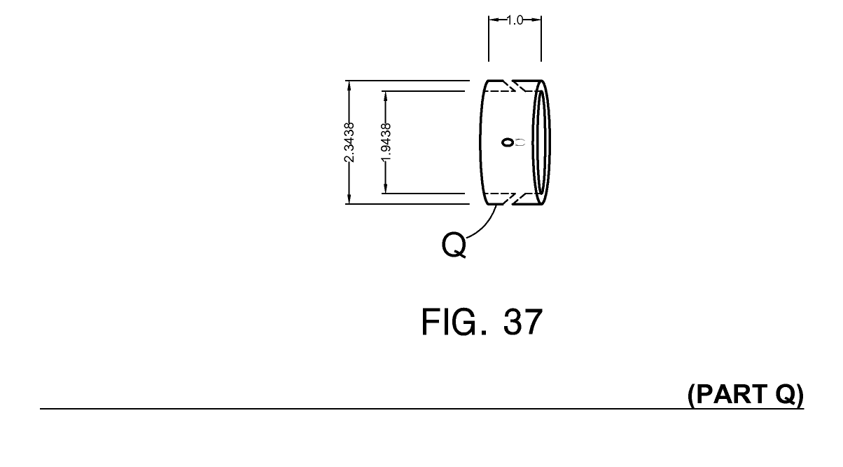

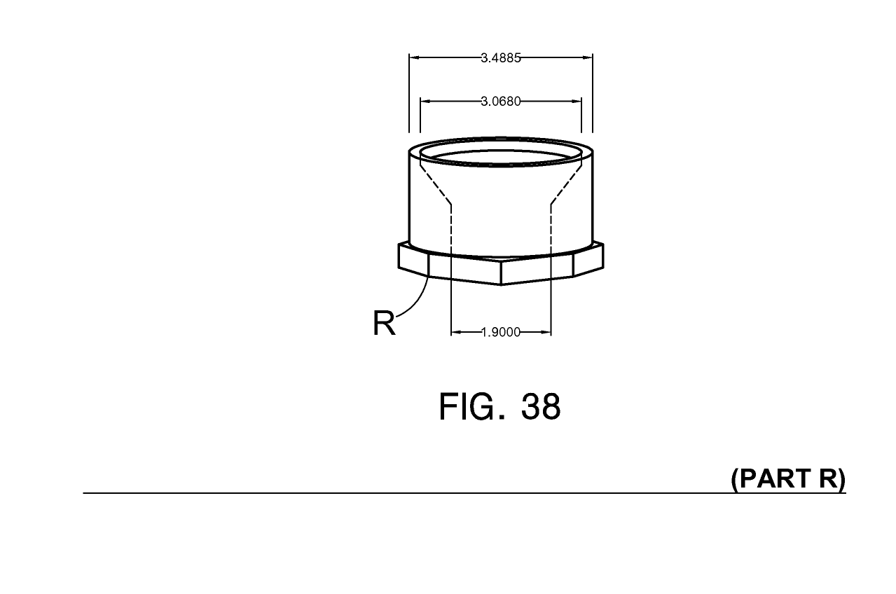

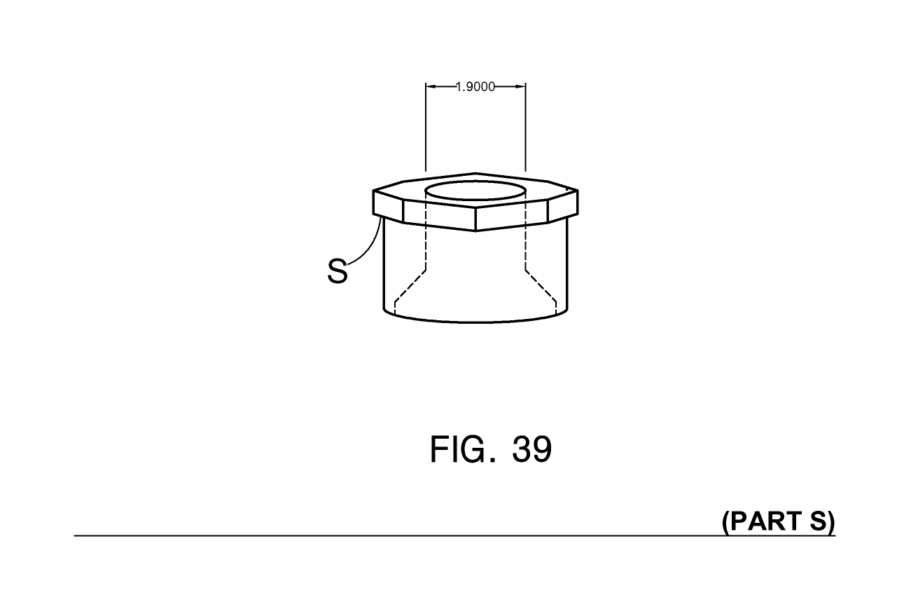

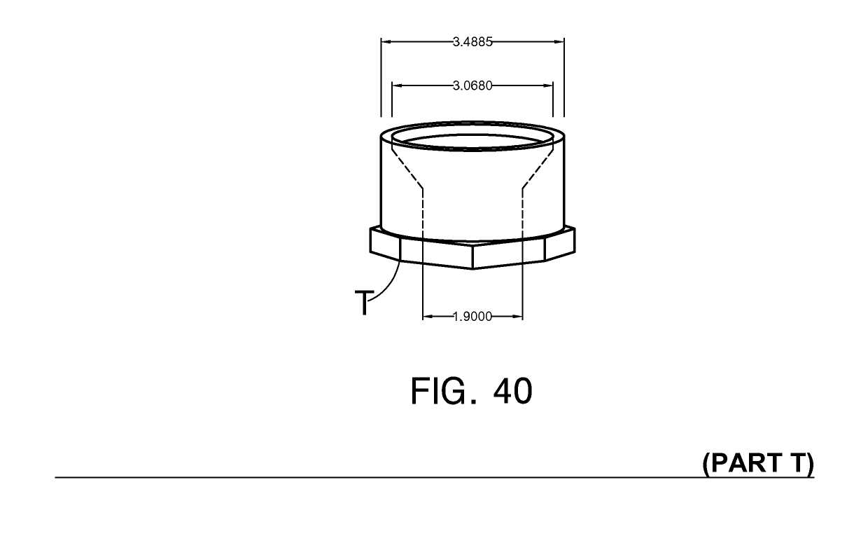

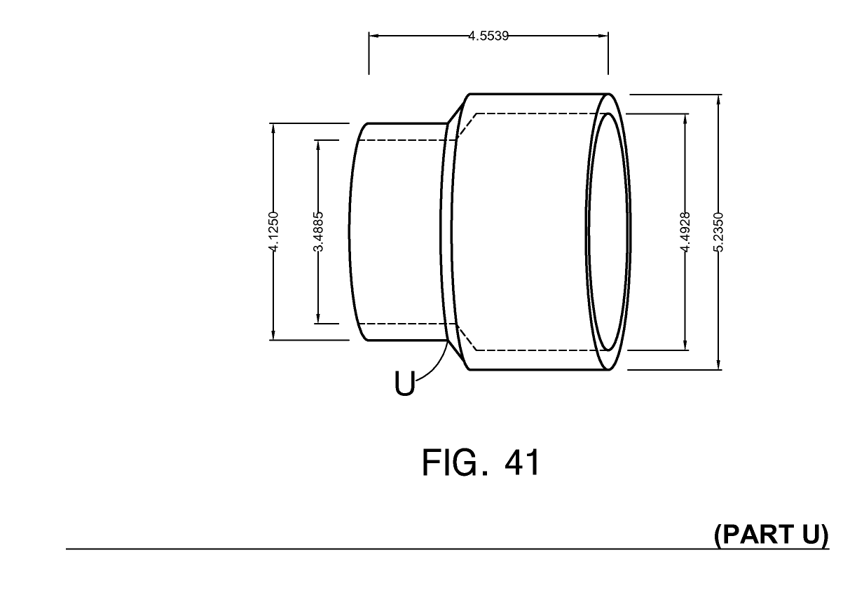

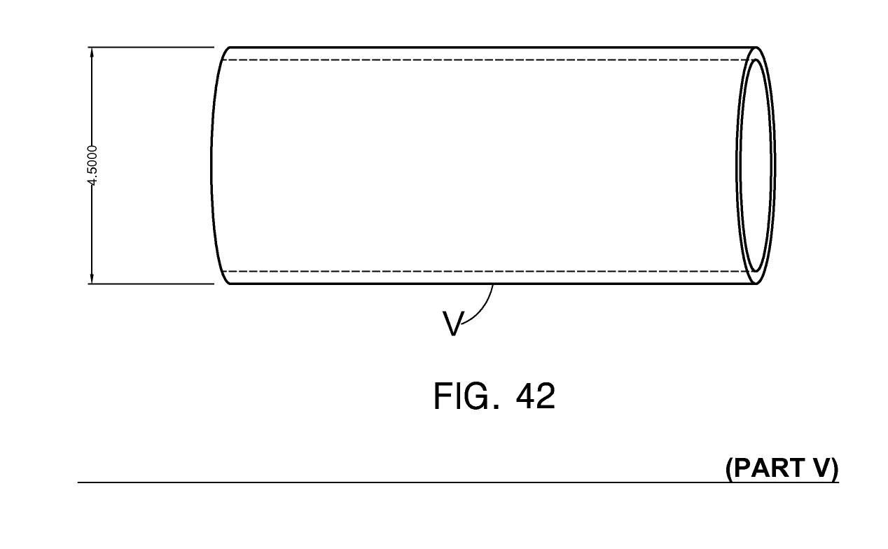

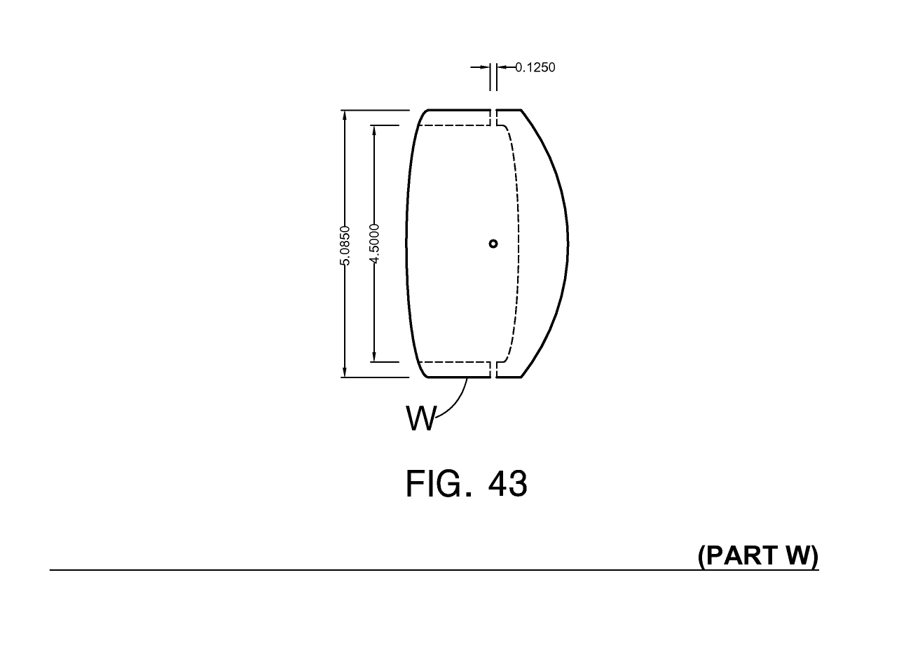

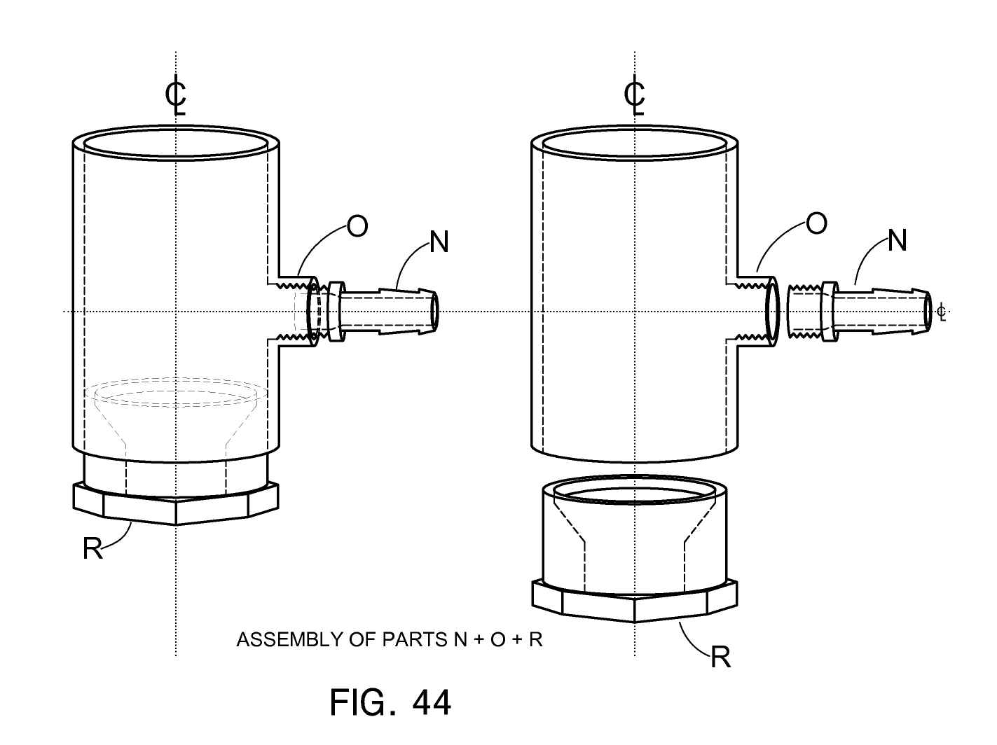

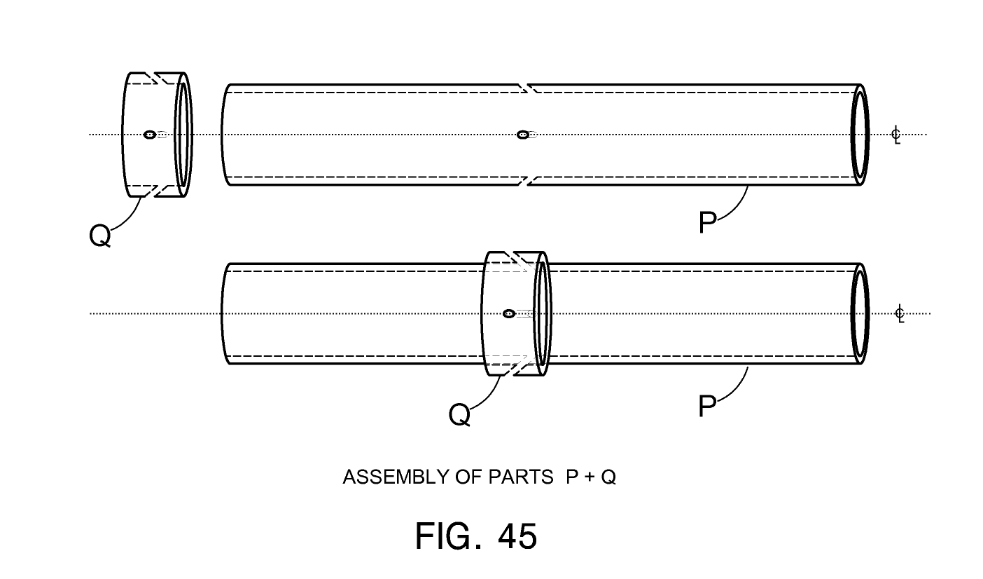

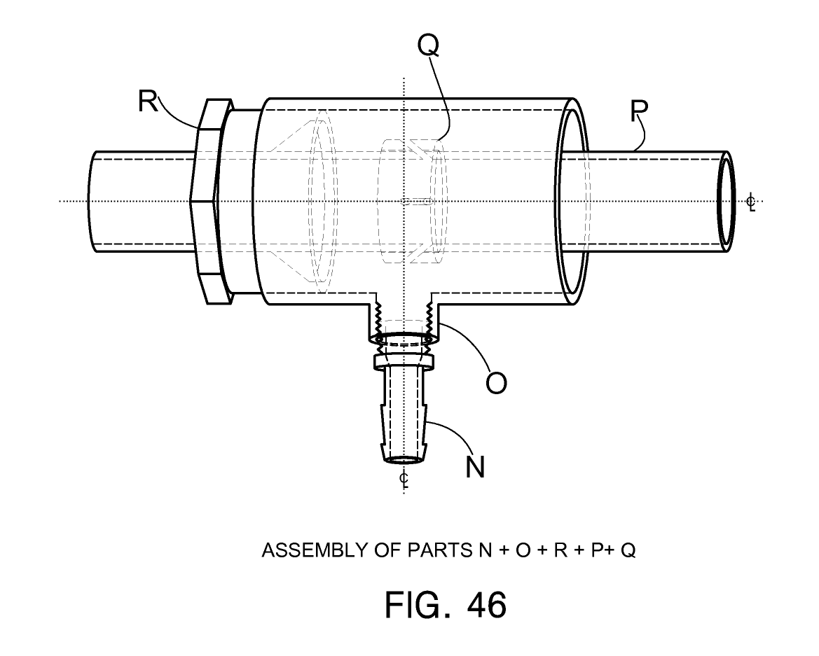

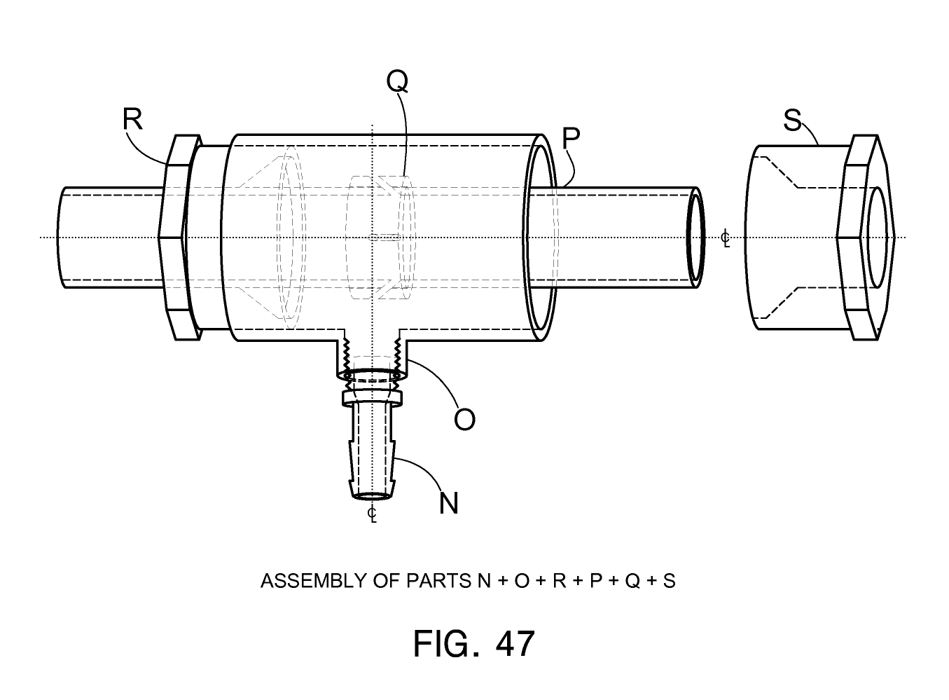

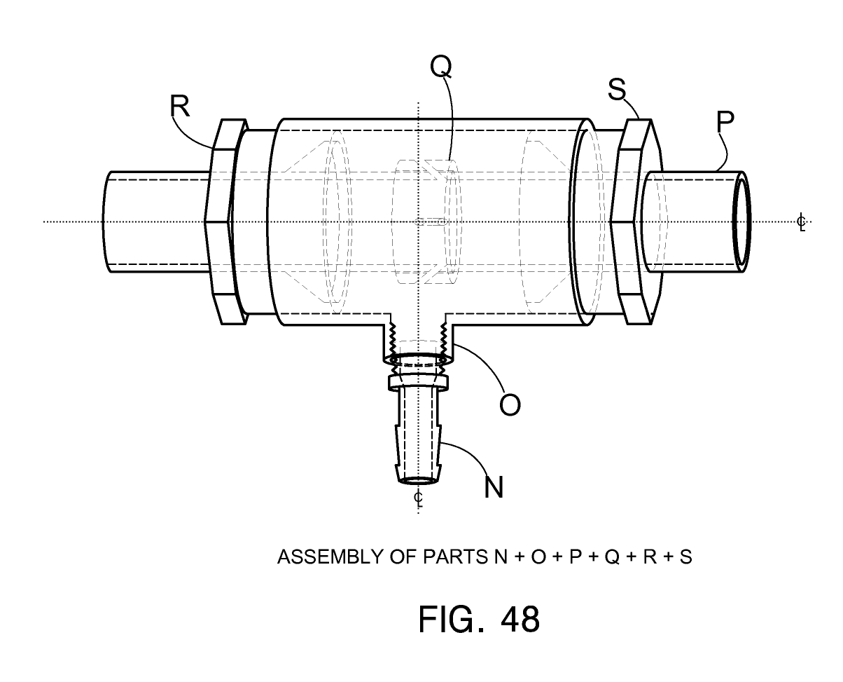

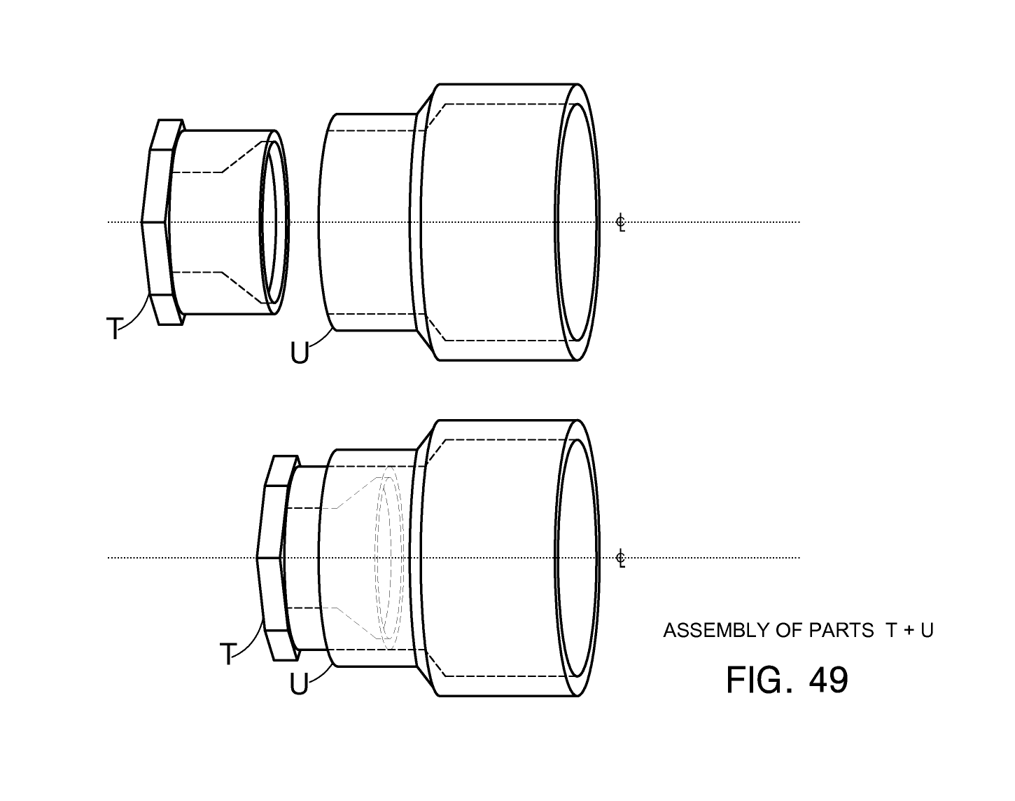

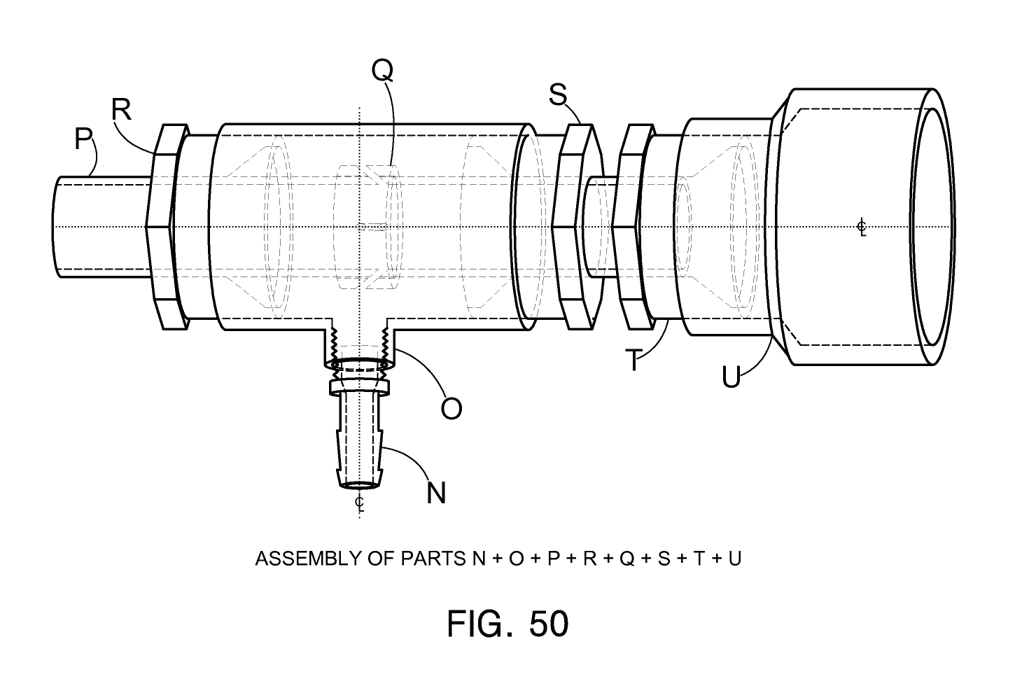

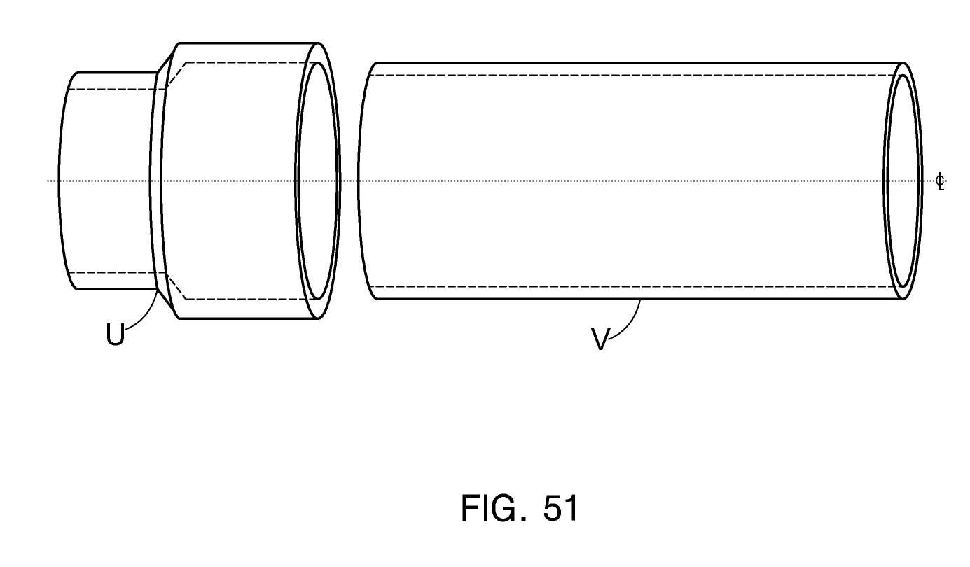

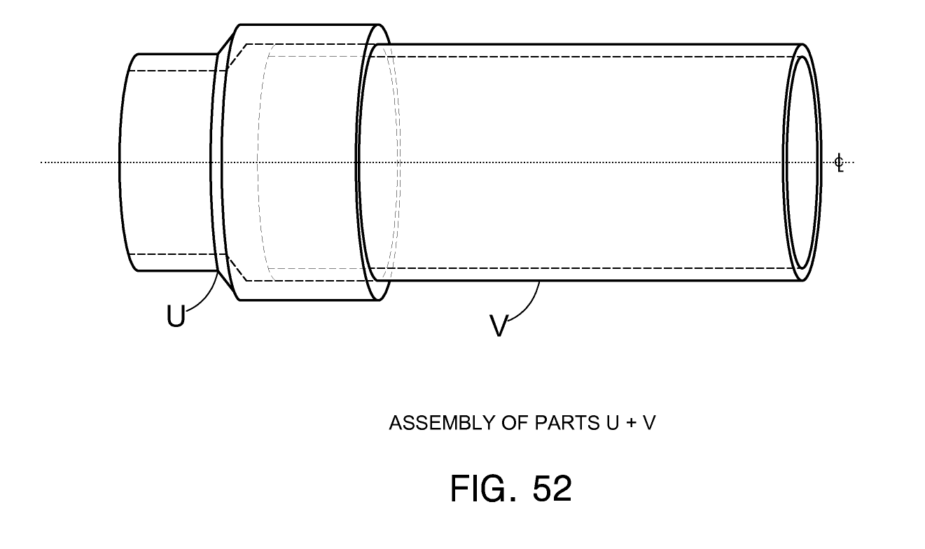

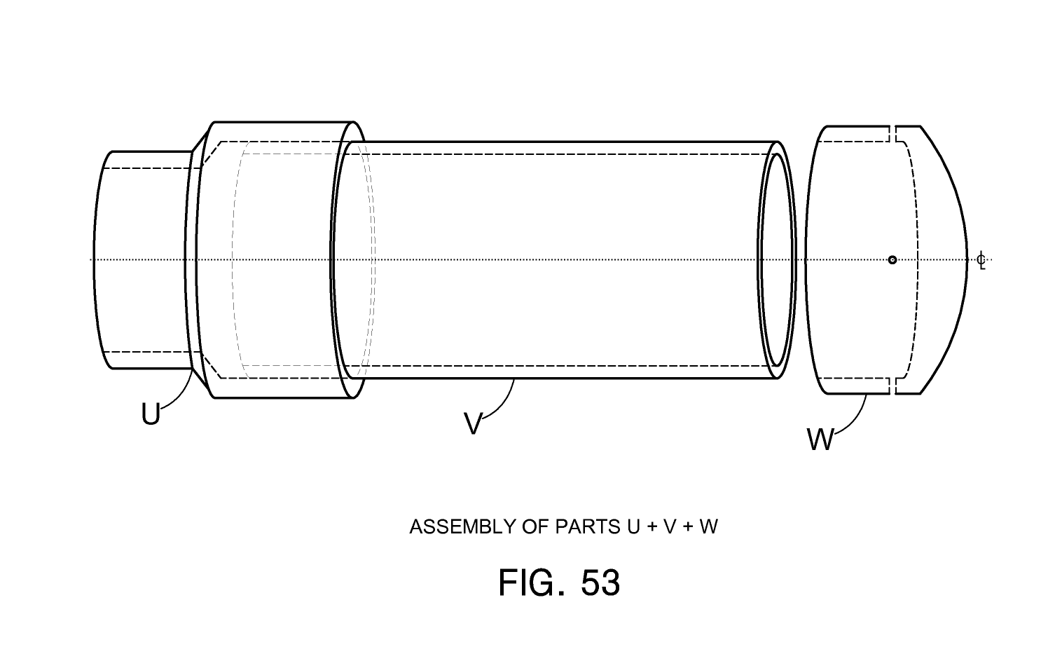

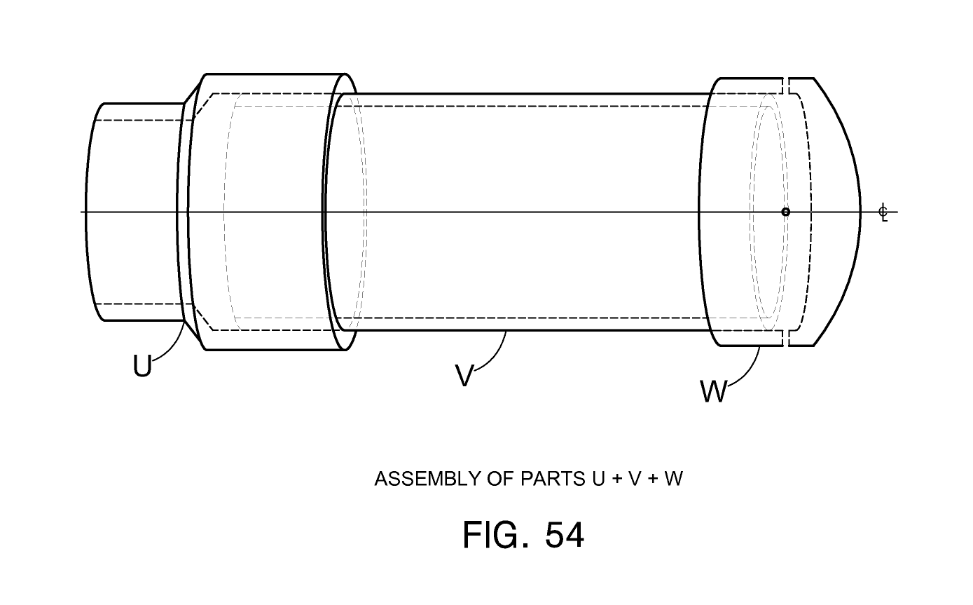

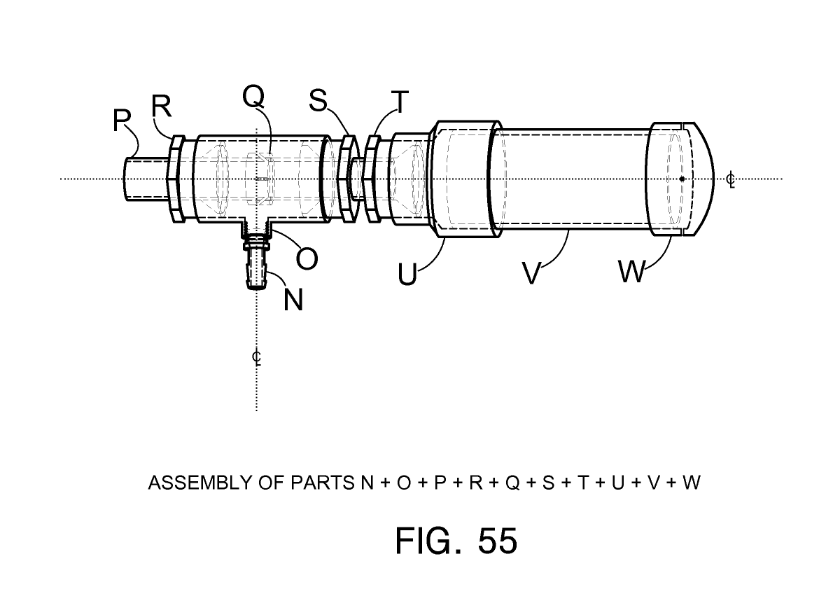

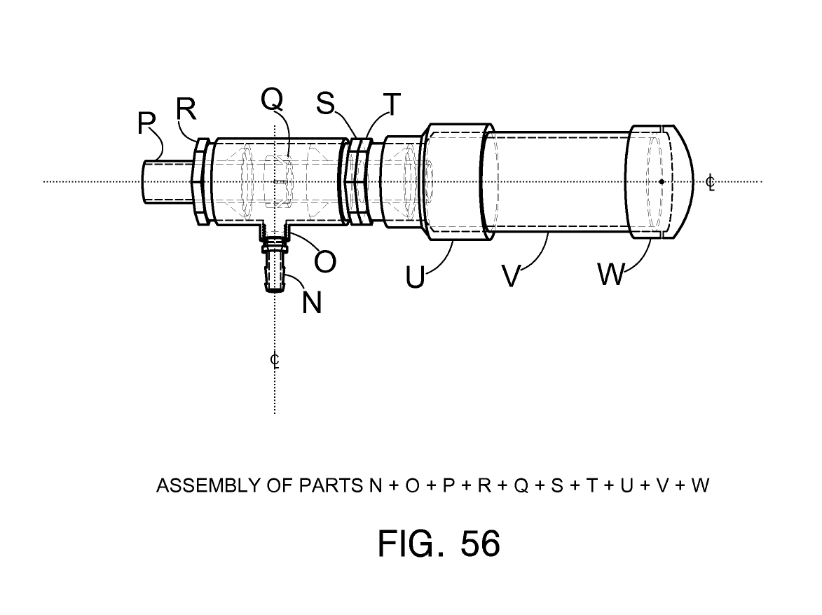

FIGS. 34 through 57, taken together, illustrate the assembly of another embodiment of the invention, wherein a hydraulic apparatus used to produce multiple jets of solution into the standpipe. In this embodiment, the apparatus comprises a length of vertical standpipe of 4-inch nominal diameter, uncapped at the bottom but capped and vented at the top, into which an intermittent low pressure jet of solution is directed upwards by means of a 1½ inch nominal diameter pipe located coaxially to the standpipe and located inferiorly to the bottom end of the standpipe (see FIG. 56).

In this embodiment, the standpipe is open to the air on the bottom as well as on the top. Initially, the standpipe is empty. The working cycle commences when solution is discharged under moderate pressure into a manifold surrounding the circumference of a section of 1½ inch nominal diameter pipe located at the bottom end of the standpipe. The solution flows into the 1½ inch nominal diameter pipe by means of holes that are oriented diagonally upward into a 1½ inch nominal diameter pipe. As more and more solution is discharged upwards under moderate pressure into the center of the 1½ inch nominal diameter pipe, the solution flows upwards into the 4-inch nominal diameter standpipe; the initial solution deposited in the standpipe is temporarily prevented from flowing downward and outward through the open bottom of the standpipe by the momentum transfer delivered to the growing column of solution within the standpipe by the later jet of solution directed vertically upward. The instant the upward jet of solution is terminated, the accumulated column of solution in the standpipe begins to fall downward and outward through the open bottom of the standpipe due to the effect of gravity. A predetermined volume of solution may thus be deposited periodically on a localized area of ground surrounding a plant whilst the tractor moves along the row of crops towing this irrigation apparatus behind.

The following is a step-by-step description of this embodiment of the invention during one working cycle.

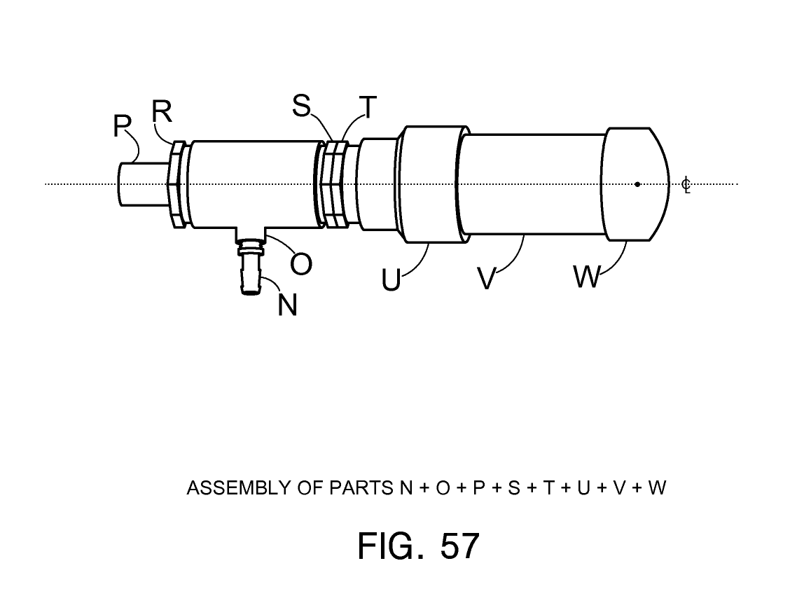

Step 1: In this embodiment, the flow of aqueous solution is delivered to the hydraulic standpipe Assembly NOPRQSTUVW (FIG. 57) by means of a flexible hose of linch (NPS) diameter; the flexible hose connects to the hydraulic standpipe Assembly NOPRQSTUVW at the barbed fitting (Part N) shown in FIG. 34.

Step 2: In an embodiment, the aqueous solution is injected into the hydraulic apparatus periodically under operating pressures ranging from 15 psi (minimum) to 80 psi (maximum); likewise, this causes the hydraulic pressure in the manifold to increase to 15 psi (minimum) to 80 psi (maximum) because there is very little loss of hydraulic pressure between the flexible hose of linch (NPS) diameter and the manifold between Part O and Assembly PQ.

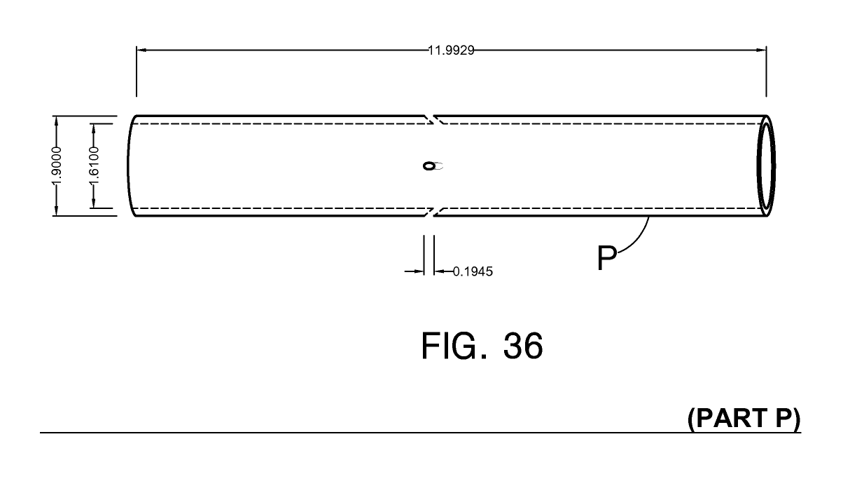

Step 3: In an embodiment, the solution flows from the manifold through the 3/16 inch diameter holes shown on FIGS. 36, 37 and 47. This causes an array of jets of solution to be directed upwards into the 1½ inch pipe (Part P).

Step 4: In an embodiment, the array of jets of solution directed upward intol-½ inch pipe (Part P) rise upward into the 4 inch diameter standpipe (Part V).

Step 5: In an embodiment, the jet streams of solution directed upward into the 1½ inch pipe (Part P) at relatively high pressure lose pressure as solution accumulates near the top of the 4-inch nominal diameter standpipe. The solution accumulates near the top of the 4-inch nominal diameter standpipe instead of the near the bottom because the column of solution is forced upwards by the momentum transfer of later aliquots of solution being added to the previously deposited aliquots of solution. Thus, the column of solution grows from the top down; the column of solution is suspended above the bottom of the 4 inch diameter standpipe by the forces associated with the ongoing momentum transfer from the jets of solution to the growing reservoir of solution in the 4-inch nominal diameter standpipe.

Step 6: In an embodiment, approximately midway through the working cycle, the hydraulic valves (Part 227) are shut off electronically as described above.

Step 7: In an embodiment, when the hydraulic valves (Part 227) are shut, the column of water in the standpipe falls to the surface of the ground (below) under the influence of gravity. The ventilation holes in the cap (Part W) prevent a partial vacuum from forming above the column of solution in the standpipe.

Step 8: In an embodiment, the column of solution in the standpipe (a volume of approximately ½ gallon) is the irrigation fluid that is delivered to the plant (below) at low flow rate yet during a short period of time.

Step 9: In an embodiment, the working cycle begins again when the hydraulic valves (Part 227) are opened.

Thus, specific embodiments of an apparatus and method for localized irrigation and application, fertilizers, herbicides, or pesticides to row crops have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. An electronically-controlled apparatus for applying an aqueous solution to row crops comprising:
an aqueous solution tank;
a master valve comprising a 3-way valve operably interconnected to said aqueous solution tank;
a pump operably interconnected to said aqueous solution tank;
a compressor operably interconnected to said pump;
a pressure reservoir operably interconnected to said compressor;
at least one irrigation valve operably interconnected to said compressor;
at least one precision hydraulic applicator;
at least one low-pressure jet integrated with said precision hydraulic applicator;
an input hose having a progressively increasing diameter, wherein said input hose is located between the output of said at least one irrigation valve and the input of said precision hydraulic applicator;
at least one timer;
at least one relay;
an electronic control subsystem comprising:
a first emitter and a second emitter;
at least one photo detector;
a phase detector; and
a controller;
wherein said optical detector, said phase detector, and said controller are all electrically interconnected;
wherein said at least one irrigation valve is electrically interconnected to said at least one timer, said at least one relay, and said electronic control subsystem;
and
a power source, wherein said power source is electrically interconnected to: said electronic control subsystem, said pump, said at least one timer, said at least one relay, said first emitter, said second emitter, and said compressor.

2. An apparatus according to claim 1, wherein said precision hydraulic applicator comprises at least 2 standpipes.

3. An apparatus according to claim 2, wherein said standpipes are arranged in series, in parallel, or any combination thereof.

4. An apparatus according to claim 1, wherein said at least one low-pressure jet has a muzzle velocity of about 7 feet per second.

5. An apparatus according to claim 1, wherein said aqueous solution comprises water, fertilizer, pesticide, herbicide, or any combination thereof.

6. An apparatus according to claim 1 wherein:
said first emitter emits radiation at a wavelength of about 670 nm; and
said second emitter emits radiation at a wavelength of about 750 nm.

7. An apparatus according to claim 1, wherein said at least one low-pressure jet comprises a manifold having a plurality of low-pressure jets.

8. An apparatus according to claim 1, wherein said precision hydraulic applicator comprises a pipe having a diameter from about 2 to 6 inches.

9. An electronically-controlled apparatus for applying an aqueous solution to row crops comprising:
an aqueous solution tank;
a master valve comprising a 3-way valve operably interconnected to said aqueous solution tank;
a pump operably interconnected to said aqueous solution tank;
a compressor operably interconnected to said pump;
a pressure reservoir operably interconnected to said compressor;
a plurality of irrigation valves operably interconnected to said compressor;
a plurality of precision hydraulic applicators, wherein each of said precision hydraulic applicators further comprises a manifold having a plurality of low-pressure jets integrated with each of said respective precision hydraulic applicators;
a plurality of input hoses having a progressively increasing diameter, wherein each of said input hoses is located between the output of a corresponding irrigation valve and the corresponding input of a precision hydraulic applicator;
a plurality of timers;
a plurality of relays;
an electronic control subsystem comprising:
a first emitter and a second emitter;
a first photo detector;
a second photo detector;
a phase detector; and
a controller;
wherein each of said photo detectors, said phase detector, and said controller are all electrically interconnected;
wherein each of said irrigation valves is electrically interconnected to a corresponding timer, a corresponding relay, and said electronic control subsystem;
and
a power source, wherein said power source is electrically interconnected to: said electronic control subsystem, said pump, said timers, said relays, said first emitter, said second emitter, and said compressor.

10. An electronically-controlled apparatus for applying an aqueous solution to row crops comprising:
an aqueous solution tank;
a master valve comprising a 3-way valve operably interconnected to said aqueous solution tank;
a pump operably interconnected to said aqueous solution tank;
a compressor operably interconnected to said pump;
a pressure reservoir operably interconnected to said compressor;
at least one irrigation valve operably interconnected to said compressor;
at least one precision hydraulic applicator;
a manifold comprising a plurality of low-pressure jets integrated with said precision hydraulic applicator;
an input hose having a progressively increasing diameter, wherein said input hose is located between the output of said at least one irrigation valve and the input of said precision hydraulic applicator;
at least one timer;
at least one relay;
an electronic control subsystem comprising:
a first emitter and a second emitter;
at least one photo detector;
a phase detector; and
a controller;
wherein said optical detector, said phase detector, and said controller are all electrically interconnected;
wherein said at least one irrigation valve is electrically interconnected to said at least one timer, said at least one relay, and said electronic control subsystem;
and
a power source, wherein said power source is electrically interconnected to: said electronic control subsystem, said pump, said at least one timer, said at least one relay, said first emitter, said second emitter, and said compressor.

11. A method of electronically and rapidly delivering an aqueous solution to a localized annulus of ground surrounding a plant, said method comprising the steps of:
(a) pumping said aqueous solution from a tank through a first pipe;
(b) reducing the hydrostatic pressure and velocity of said aqueous solution by continuing to flow said aqueous solution into a second pipe having a greater diameter than said first pipe;
(c) maintaining laminar flow of said aqueous solution simultaneously with step (b);
(d) streaming a plurality of aliquots of said aqueous solution in an upward direction from the output of said second pipe into the center of) a standpipe;
(e) terminating the streaming of step (d); and
(f) delivering said aqueous solution at a low velocity from said standpipe to the target area with a specified wetting pattern.

12. A method according to claim 1, wherein step (d) further comprises suspending progressive columns of aqueous solution in said standpipe.

13. A method according to claim 1, wherein said streaming in step (d) alternatively comprises injecting a plurality of simultaneous streams of said aqueous solution from a series of low pressure jets into said standpipe.

14. A method according to claim 1, wherein said steps (a) to (f) are completed within a total cycle timeframe of about 1 second.

15. A method according to claim 1, wherein step (f) produces a resulting oval wetting pattern of about 3 inches by 6 inches.

16. A method according to claim 1, wherein said low velocity in step (f) is less than about 14 feet per second.

17. A method according to claim 1, wherein said low velocity in step (f) is an average of about 13 feet per second.

18. A method according to claim 1, wherein said aqueous solution comprises water, fertilizer, pesticide, herbicide, or any combination thereof.

19. A method of automatically sensing and distinguishing a plant from soil, said method comprising the steps of:
(a) emitting radiation of a first wavelength from a first emitter;
(b) emitting radiation of a second wavelength from a second emitter;
(c) modulating said first and second emitters at a high rate of speed;
(d) shifting the modulation of said first emitter by approximately 90 degrees relative to said second emitter;
(e) focusing said first and second emitters on a target to reflect said first and second emitter wavelengths;
(f) using a photo receptor to intercept the reflected radiation wavelengths from step (e);
(g) calculating a ratio value from step (f) of said first and second reflected wavelengths;
(h) converting the ratio value of step (g) to a phase;
(i) comparing the phase of step (h) to an initial reference phase of said first or said second emitter; and
(j) processing the output of step (i) via a digital controller to electronically determine the presence of a plant or soil.

20. A method according to claim 19, wherein said first wavelength is about 670 nm and said second wavelength is about 750 nm.
